SEQUENCE LISTING (27 sequences)

SEQ ID NO: 1 - protein, 143 aa, synthetic construct
  REGION 1..143: note = Description of sequence: VHH peptide
  REGION 1..143: note = Name of sequence: EFH-1F2
EVQLVESGGRLVQTGGSLRLSCAASGRIFSSYAMGWFRQAPGKEREFVAAVSMGGRTIYGDSVKGRFTISRDNAKNTVWLQMNSLKPEDTAVYYCAQGVYSVVPGTYDYWGQGTQVTVSSAAADYKDDDDKGAAHHHHHHGAA

SEQ ID NO: 2 - protein, 151 aa, synthetic construct
  REGION 1..151: note = Description of sequence: VHH peptide
  REGION 1..151: note = Name of sequence: EFH-1B2
EVQLVESGGGLVQAGGSLRLSCAASGRTFSSYHMGWFRQAPGKEREFIAAISWSDSSTYYADSVKGRFTISRDNAKNTRYLQMNSLKPEDTAVYYCAAASRRTFVSGSAYYRDDSYDYWGQGTQVTVSSAAADYKDDDDKGAAHHHHHHGA

SEQ ID NO: 3 - protein, 144 aa, synthetic construct
  REGION 1..144: note = Description of sequence: VHH peptide
  REGION 1..144: note = Name of sequence: EFH-1D1
EVQLVESGGGLVQAGGSLRLSCAASGFTFSSYAMGWFRKAPGKEREFVAAISMSGGSTYYADSVKGRFTISRENAKNTVYLQMNSLKPEDTAVYYCAAGVYSLVASEYDYWGQGTQVTVSSAAADYKDDDDKGAAHHHHHHGAA

SEQ ID NO: 4 - protein, 144 aa, synthetic construct
  REGION 1..144: note = Description of sequence: VHH peptide
  REGION 1..144: note = Name of sequence: EFH-1G4
EVQLVESGGGLVQAGGSLRLSCAASGFTFSSYAMGWFRQAPGKEREFVAAISWSGGSTYYADSVKGRFTISRENAENTVYLQMNSLKIEDTAVYYCAAGVYSLVASEYDYWGQGTQVTVSSAAADYKDDDDKGAAHHHHHHGAA

SEQ ID NO: 5 - protein, 144 aa, synthetic construct
  REGION 1..144: note = Description of sequence: VHH peptide
  REGION 1..144: note = Name of sequence: EFH-1B3
EVQLVESGGELVQAGGSLRLSCAASGFTFSSYAMGWFRQAPGKEREFVAAISHSGGSTYYADSVKGRFTISRDNAKNTAYLQMNSLKIEDTAVYYCASGVYSLVASEYDYWGQGTQVTVSSAAADYKDDDDKGAAHHHHHHGAA

SEQ ID NO: 6 - protein, 144 aa, synthetic construct
  REGION 1..144: note = Description of sequence: VHH peptide
  REGION 1..144: note = Name of sequence: EFH-1F4
EVQLVESGGGLVQAGGSLRLSCAASGLTFGSYAMGWFRQAPGKEREFVATISRSGGSTYYADAVKGRFTISRDNAKNTVYLQMNSLKPEDTAVYYCAAGVLAVVADPYDYWGQGTQVTVSSAAADYKDDDDKGAAHHHHHHGAA

SEQ ID NO: 7 - protein, 144 aa, synthetic construct
  REGION 1..144: note = Description of sequence: VHH peptide
  REGION 1..144: note = Name of sequence: EFH-1E4
EVQLVESGGGLVQPGGSLRLSCAASGSIANIVAMDWYRQAPEKQRELVASITSSGGTSYADSVQGRFAISRDNAKNTVYLQMNRLKPEDTAVYYCNGFIRSSNGGRLNGYWGQGTQVTVSSAAADYKDDDDKGAAHHHHHHGAA

SEQ ID NO: 8 - protein, 140 aa, synthetic construct
  REGION 1..140: note = Description of sequence: VHH peptide
  REGION 1..140: note = Name of sequence: EFH-1A1
EVQLVESGGGLVQTGGSLRLSCAASGIIFSTKTMGWYRQAPGKQREWVATLTSGGSPNYADSLKGRFTISRDNLKNMVYLQMNNLKPEDTAVYYCAAQRADSWSTSYRGQGTQVTVSSAAADYKDDDDKGAAHHHHHHGA

SEQ ID NO: 9 - protein, 146 aa, synthetic construct
  REGION 1..146: note = Description of sequence: VHH peptide
  REGION 1..146: note = Name of sequence: EFH-1B8
EVQLVESGGGLVQAGGSLRLSCAASGSVFSINVMGWYRQAPGKQRELVAAITRGGSTNYGDSVKGRFTISRDNAKNMVYLQMNSLKPEDTAVYYCAADPGTWLAYGGVEYDYWGQGTQVTVSSAAADYKDDDDKGAAHHHHHHGAA

SEQ ID NO: 10 - protein, 146 aa, synthetic construct
  REGION 1..146: note = Description of sequence: VHH peptide
  REGION 1..146: note = Name of sequence: EFH-1E12
EVQLVESGGGLVQAGGSLRLSCAASGSTFSINVMGWYRQAPGKQRELVAAITRGGSTNYADSVKGRFTISRDNAKNTVYLQMNSLKPEDTAVYNCAADPGTWLAYGGVEYDYWGQGTQVTVSSAAADYKDDDDKGAAHHHHHHGAA

SEQ ID NO: 11 - protein, 144 aa, synthetic construct
  REGION 1..144: note = Description of sequence: VHH peptide
  REGION 1..144: note = Name of sequence: EFH-1C5
EVQLVESGGGLVQAGGSLRLSCVASGRTFSSYGMGWFRQAPGKEREFVAAVSVSGSSTYYADTVKGRVTISRDNVKNTVYLQMNSLKAEDTAVYYCAAGVYGGVGTLYDYWGRGTQVTVSSAAADYKDDDDKGAAHHHHHHGAA

SEQ ID NO: 12 - protein, 144 aa, synthetic construct
  REGION 1..144: note = Description of sequence: VHH peptide
  REGION 1..144: note = Name of sequence: EFH-1F8
EVQLVESGGGLVQAGGSLTLSCAGSGRTFNNYGVGWFRQAPGKEREFVAAISQGRSSTYYSDSVKGRFTVSSDNSKNTVYLQMNSLKPEDTAVYYCAAGIYGRVSSLYDYWGQGTQVTVSSAAADYKDDDDKGAAHHHHHHGAA

SEQ ID NO: 13 - protein, 148 aa, synthetic construct
  REGION 1..148: note = Description of sequence: VHH peptide
  REGION 1..148: note = Name of sequence: EFH-1G10
EVQLVESGGGLVQAGGSLRLSCEATGRTFSDYAIGWFRQAPGKEREFVAAIRMNDGRTYYADSVKGRFTISRDYAENTVYLQMDSLKPEDTAVYYCALDLYAGALTTTRAEYHYWGQGTQVTVSSAAADYKDDDDKGAAHHHHHHGAA

SEQ ID NO: 14 - protein, 141 aa, synthetic construct
  REGION 1..141: note = Description of sequence: VHH peptide
  REGION 1..141: note = Name of sequence: EFH-2E2
EVQLVESGGGLVQPGGSLRISCAASGSIFSMNVMGWYRQAPGKQREFVAGIASGDNARYADSVKGRFTISRDNAKNTVYLQMNSLKSEDTAVYYCGAATAWGSRVGSWGQGTQVTVSSAAADYKDDDDKGAAHHHHHHGAA

SEQ ID NO: 15 - protein, 141 aa, synthetic construct
  REGION 1..141: note = Description of sequence: VHH peptide
  REGION 1..141: note = Name of sequence: EFH-2B5
EVQLVESGGGLVQPGGSLRISCAASGSIFSMNVMGWYRQAPGKQREFVAGITSGDNARYADSVKGRFTISRDNAKNTVYLQMNSLKSEDTAVYYCGAATAWGSRVGSWGQGTQVTVSSAAADYKDDDDKGAAHHHHHHGAA

SEQ ID NO: 16 - protein, 141 aa, synthetic construct
  REGION 1..141: note = Description of sequence: VHH peptide
  REGION 1..141: note = Name of sequence: EFH-2B1
EVQLVESGGGLVQPGGSLRLSCAASGSIFSINVMGWYRQAPGKQREFVAGIASGDNTHYRDSVKGRFTISRDNAKNTVYLQMNSLKSEDTAVYYCGAATAWGSRVHSWGQGTQVTVSSAAADYKDDDDKGAAHHHHHHGAA

SEQ ID NO: 17 - protein, 141 aa, synthetic construct
  REGION 1..141: note = Description of sequence: VHH peptide
  REGION 1..141: note = Name of sequence: EFH-2B4
EVQLVESGGGLVQPGGSLRLSCAASGSIFSINVMGWYRQAPGKQREFVAGITSGDNTRYRDSVKGRFTISRDNAKNTVYLQMNSLKSEDTAVYYCGAATAWGSRVDSWGQGTQVTVSSAAADYKDDDDKGAAHHHHHHGAA

SEQ ID NO: 18 - protein, 141 aa, synthetic construct; identical to SEQ ID NO: 17
  REGION 1..141: note = Description of sequence: VHH peptide
  REGION 1..141: note = Name of sequence: EFH-2F6

SEQ ID NO: 19 - protein, 141 aa, synthetic construct
  REGION 1..141: note = Description of sequence: VHH peptide
  REGION 1..141: note = Name of sequence: EFH-2D2
EVQLVESGGGLVQPGGSLRLSCAASGSIFSINVMGWYRQAPGKQREFVAGITSGDNTRYRDSVKGRFTISRDNAKNTVYLQMNSLESEDTAVYYCGAATAWGSRVGSWGQGTQVTVSSAAADYKDDDDKGAAHHHHHHGAA

SEQ ID NO: 20 - protein, 141 aa, synthetic construct; identical to SEQ ID NO: 19
  REGION 1..141: note = Description of sequence: VHH peptide
  REGION 1..141: note = Name of sequence: EFH-2C6

SEQ ID NO: 21 - protein, 141 aa, synthetic construct
  REGION 1..141: note = Description of sequence: VHH peptide
  REGION 1..141: note = Name of sequence: EFH-2G5
EVQLVESGGGLVQPGGSLRLSCAASGSIFSINVMGWYRQAPGKQREFVAGITSGDNTRYRDSVKGRFTISRDNAKNTVYLQMNSLKSEDTAVYYCGAATAWGSRVGSWGQGTQVTVSSAAADYKDDDDKGAAHHHHHHGAA

SEQ ID NO: 22 - protein, 141 aa, synthetic construct; identical to SEQ ID NO: 21
  REGION 1..141: note = Description of sequence: VHH peptide
  REGION 1..141: note = Name of sequence: EFH-2F11

SEQ ID NO: 23 - protein, 141 aa, synthetic construct
  REGION 1..141: note = Description of sequence: VHH peptide
  REGION 1..141: note = Name of sequence: EFH-2A7
EVQLVESGGGLVQAGGSLRLSCAASGRTFSNNDLAWFRKAPGKEREFVARINWTGNLIYYQGSVKGRFTISRDNAKNTVYLQMNSLKPEDTAVYYCAARGVRGSYDYWGQGTQVTVSSAAADYKDDDDKGAAHHHHHHGAA

SEQ ID NO: 24 - protein, 144 aa, synthetic construct
  REGION 1..144: note = Description of sequence: VHH peptide
  REGION 1..144: note = Name of sequence: EFH-2D9
EVQLVESGGGLVQAGGSLRLSCAGSGRTFSNYGTGWFRQAPGKEREFVAAINRRGSDTYYSDSVKGRFTVSRDNNKNTVYLQMNSLKPEDTAVYYCAAGIYGRVSSLYDYWGQGTQVTVSSAAADYKDDDDKGAAHHHHHHGAA

SEQ ID NO: 25 - protein, 146 aa, synthetic construct
  REGION 1..146: note = Description of sequence: VHH peptide
  REGION 1..146: note = Name of sequence: EFH-2D7
EVQLVESGGGLVQAGGSLRLSCAASGRTFSAYAMGWFRQAPGKEREFVAAITWNGRSTYYTDSVKGRFTISRDNAKNTVYLQMNSLKPEDTAVYYCAAGRISRRLVAGDFDSWGQGTQVTVSSAAADYKDDDDKGAAHHHHHHGAA

SEQ ID NO: 26 - protein, 157 aa, synthetic construct
  REGION 1..157: note = Description of sequence: VHH peptide
  REGION 1..157: note = Name of sequence: EFH-2E12
EVQLVESGGGLVQAGGSLRLSCAASGRTVSTYAMGWFRQAPGKEREFVAHINYSGGTTNYADSVKGRFTISRDDTKLTISQRASSLKATLYLQMDSLKAEDTAIYYCAANPKGTWFRPSDYNYWGQGTQVTVSSAAADYKDDDDKGAAHHHHHHGAA

SEQ ID NO: 27 - protein, 145 aa, synthetic construct
  REGION 1..145: note = Description of sequence: VHH peptide
  REGION 1..145: note = Name of sequence: EFH-2E10
EVQLVESGGDLVQPGGSLRLSCAASGFIFSAYAMSWVRQAPGKGLEWVSGINAGGFTRNYADSVKGRFTISRDNDKNAVYLQMNSLKPEDTAVYYCAKGSMWDRRGKSYDNLGQGTQVTVSSAAADYKDDDDKGAAHHHHHHGAA

(19) United States
(12) Patent Application Publication
(10) Pub. No.: US 2025/0304661 A1
(43) Pub. Date: Oct. 2, 2025

(54) VHH ANTIBODIES TARGETING FIMH AND METHODS OF USING THE SAME

(71) Applicant: BiomEdit, LLC, Fishers, IN (US)

(72) Inventors: Dharanesh Mahimapura Gangaiah, Fishers, IN (US); Arvind Kumar, Fishers, IN (US)

(21) Appl. No.: 18/694,164

(22) PCT Filed: Sep. 22, 2022
(86) PCT No.: PCT/US2022/044361

§ 371 Date: Mar. 21, 2024

Related U.S. Application Data
(60) Provisional application No. 63/247,363, filed on Sep. 23, 2021.

(51) Int. Cl. C07K 16/12

(52) U.S. Cl. CPC C07K 16/1232; C07K 2317/33; C07K 2317/569; C07K 2317/92

(57) ABSTRACT

One or more antibodies, particularly nanobodies or VHH single domain antibodies, directed against one or more FimH targets with roles in E. coli colonization in animals are provided. The nanobodies are useful in reducing or inhibiting E. coli colonization or infection. Methods of treating and/or preventing E. coli infection and conditions related thereto are provided.

### CROSS-REFERENCE TO RELATED APPLICATIONS

This application is the National Stage of International Application No. PCT/US2022/044361, filed Sep. 22, 2022, which claims priority to Provisional Patent Application No. 63/247,363, filed Sep. 23, 2021, the contents of each are hereby incorporated by reference herein.

### FIELD OF THE INVENTION

The present invention relates to one or more antibodies, particularly nanobodies or VHH single domain antibodies, directed against one or more FimH targets.

### REFERENCE TO A SEQUENCE LISTING

The instant application contains a Sequence Listing encoded in XML which was filed electronically by EFS-web and is hereby incorporated by reference in its entirety. Said XML format Sequence Listing, created on Sep. 22, 2022, is named “2950-14-PCT_ST26.xml” and is 49,027 bytes in size

### BACKGROUND OF THE INVENTION

FimH is a 2-domain protein composed of an N-terminal, mannoside-binding lectin domain (FimH_{L}) and a C-terminal pilin domain (FimH_{P}). FimH is a virulence factor and a therapeutic target for urinary tract infections (UTI) and gastrointestinal diseases such as, for example, Crohn's Disease and Ulcerative Colitis. For example, its has been discovered that FimH targets are located on type 1 pili of uropathogenic E. coli (UPEC), and play an integral role in the pathogenesis of UPEC. See, e.g. Mydock-McGrane, et al. Rational design strategies for FimH antagonists: new drugs on the horizon for urinary tract infection and Crohn's disease. Expert Opin Drug Discov. 2017 July; 12(7):711-731.

E. coli is a bacteria that is commonly found in the lower intestines of most animals. Most strains of E. coli are a harmless part of the gut flora. However, certain strains of E. coli can cause serious food poisoning and remain a major public health concern. In addition, adherent invasive Escherichia coli (AIEC) has been linked to Crohn's disease, while diffusely adherent E. coli (DAEC) has been associated with ulcerative colitis. E. coli is also the leading cause of urinary tract infections (UTIs), uncluding cystitis and pyelonephritis in animals. More than 700 serotypes of E. coli have been identified. Shiga toxin-producing E. coli (STEC) is one strain known to cause severe gastroenteritis. Uropathogenic E. coli (UPEC) strains possess the ability to adhere to host epithelial cells in the urinary tract.

Although most E. coli infections are acute and self-limiting, recurring UTIs, Crohn's Disease and Ulcerative Colitis are chronic, debilitating conditions that result in high medical care costs and impaired quality of life.

Thus, it is apparent that conditions related to E. coli remain a major issue. There is a long felt need for validated and effective interventions for control of E. coli infections. Development of a safe an effective preventative treatment therapy that targets E. coli overgrowth is needed. Targeting one or more E. coli targets to reduce or inhibit E. coli colonization is proposed to provide an effective intervention. The present invention is directed to this unmet need. Antibodies, particularly nanobodies which are single domain antibodies with high affinity and specificity, directed against one or more targets with key roles in E. coli colonization, provide a useful and applicable approach.

### SUMMARY OF THE INVENTION

The present invention provides novel domain antibodies, particularly nanobodies, directed against targets with key roles in E. coli colonization.

In embodiments of the invention, nanobodies directed against FimH_SI and FimH_ST antigens are provided. VHH sequences for these antibodies are provided.

The nanobodies of the present invention are useful in reducing or blocking E. coli colonization or infection. The invention relates to novel VHH single domain antibodies, and methods for reducing or inhibiting E. coli colonization or infection, reducing bacterial gastroenteritis, Crohn's Disease, Ulcerative Colitis and urinary tract infections, and providing immunity against E. coli infections in animals, including humans.

In an embodiment of the invention, combinations of one or more nanobodies directed against FimH_SI and FimH_ST antigens are provided.

In another embodiment, the antibodies directed individually to each of FimH_SI and FimH_ST are capable of binding to their specific target protein and neutralizing or inhibiting the activity of their target.

In one embodiment, the nanobody of the invention comprises a heavy chain variable region VHH sequence as set out in the figures herein, wherein the nanobody comprises an amino acid sequence having at least 80%, at least 90%, at least 95%, at least 98%, at least 99% amino acid identity to a VHH sequence as set out herein. Any such nanobody is capable of binding specifically to the applicable antigen.

The invention provides antibodies specifically directed against FimH antigens for diagnostic and therapeutic purposes. In particular, antibodies specific for each FimH antigen are provided, wherein said antibodies recognize and are capable of binding E. coli antigens.

The antibodies of the present invention have diagnostic and therapeutic use in E. coli infections and colonization, including modulating the immune response of an animal to E. coli and modulating the infection and colonization of E. coli in an animal. The antibodies of the invention are applicable in characterizing and in modulating the activity of E. coli proteins. The antibodies of the invention are applicable in modulating the activity of E. coli proteins and acting as a prophylactic or therapeutic to prevent or inhibit E. coli, including E. coli infection and/or colonization.

The antibodies of the present invention have diagnostic and therapeutic use in E. coli infections and colonization, including modulating the immune response of an animal to E. coli and modulating the infection and colonization of E. coli in an animal. The antibodies of the invention are applicable in characterizing and in modulating the activity of one or more or any of E. coli proteins. The antibodies of the invention are applicable in modulating the activity of one or more or any of E. coli proteins and acting as a prophylactic or therapeutic to prevent or inhibit E. coli, including E. coli infection and/or colonization.

The antibodies of the present invention have diagnostic and therapeutic use in bacterial gastroenteritis in animals, including humans. The antibodies of the invention are applicable in characterizing and in modulating the activity of one or more E. coli proteins and thereby reducing or alleviating bacterial gastroenteritis. The antibodies of the invention are applicable in characterizing and in modulating the activity of one or more of E. coli proteins. The antibodies of the invention are applicable in modulating the activity of one or more E. coli proteins and acting as a prophylactic or therapeutic to prevent or alleviate bacterial gastroenteritis in animals, including humans.

Methods are provided for identifying or characterizing E. coli, such as in an animal infected or colonized with E. coli utilizing one or more of the nanobodies provided herein and directed against E. coli proteins.

Methods are provided for inhibiting E. coli bacteria, such as in an animal infected or colonized with E. coli, utilizing one or more of the nanobodies provided herein and directed against E. coli proteins. Methods are provided for inhibiting E. coli bacteria, such as in an animal infected or colonized with E. coli, comprising administering to the animal one or more of the nanobodies provided herein and directed against E. coli proteins.

In further embodiments, the invention provides an isolated nucleic acid which comprises a sequence encoding a VHH polypeptide described herein, particularly a nanobody provided herein and directed against FimH. In an embodiment, the invention includes nucleic acid encoding one or more nanobody, including nanobody amino acid sequence disclosed and described herein.

The present invention also relates to a recombinant DNA molecule or cloned gene, or a degenerate variant thereof, which encodes an antibody of the present invention: preferably a nucleic acid molecule, in particular a recombinant DNA molecule or cloned gene, encoding the antibody VH. particularly the CDR region sequences, which is capable of encoding a heavy chain sequence described and as set out herein.

In accordance with the invention, methods for treatment, alleviation or modulation of Crohn's Disease, Ulcreative Colitis, or urinary tract infection or colonization due to E. Coli, comprising administering antibodies of the invention or pharmaceutical compositions thereof are provided herein.

Thus, in an embodiment of the invention the nanobodies may be administered alone or in combination with other treatments, therapeutics or agents, either simultaneously or sequentially dependent upon the condition to be treated.

The invention includes compositions and or kits, comprising one or more nanobodies of the invention together with one or more immunomodulatory or immunogenic or antibacterial proteins or peptides. The compositions include pharmaceutical compositions and immunological compositions. The nanobodies or compositions of the invention may be administered systemically or in a targeted fashion, including administration to an affected organ or organ of interest, such as to the gastrointestinal tract.

The nanobodies of the present invention, and in a particular embodiment one or more nanobody having sequence as set out herein, or active fragments thereof, and recombinant or synthetic nanobodies derived therefrom, particularly comprising the heavy chain CDR region sequences of the nanobodies can be prepared in pharmaceutical compositions, including a suitable vehicle, carrier or diluent, or including an adjuvant and/or immune modulator, for administration. Such pharmaceutical compositions may also include means for modulating the half-life of the nanobodies or fragments by methods known in the art such as pegylation.

Pharmaceutical compositions or immunogenic compositions of the invention may further comprise additional antibodies or therapeutic agents. In an aspect, such other agents or therapeutics may be selected from anti-bacterial agents or immune modulators or anti-inflammatory agents. Pharmaceutical compositions or immunological compositions may comprise a combination of one or more, two or more, three or more or four or more or five unique nanobodies as set out and provided herein. Compositions may comprise a combination of nanobodies directed against FimH proteins, particularly a combination comprising each of a nanobody described herein specific for protein. Various such combinations are contemplated herein.

The diagnostic utility of the present invention extends to the use of the nanobodies of the present invention in assays to characterize cellular samples or to screen for E. coli infection or colonization, including in vitro and in vivo diagnostic assays. Nanobodies of the invention may carry a detectable or functional label. The specific binding members may carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re. In an aspect, the label may be an enzyme, including wherein detection may be accomplished by any of the presently utilized colorimetric, spectrophotometric, fluorospectrophotometric, amperometric or gasometric techniques known in the art.

Immunoconjugates or antibody fusion proteins of the present invention, wherein nanobodies of the present invention are conjugated or attached to other molecules or agents further include, but are not limited to nanobody(ies) conjugated to a immunomodulator, cytokine, cytotoxic agent, antibacterial agent, antibiotic or drug.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1: Demonstrates the immune response of llamas SNL168 (A) and SNL169 (B) to FimH_SI.

FIG. 2: Demonstrates the immune response of llamas SNL168 (A) and SNL169 (B) to FimH_ST.

FIG. 3: A) Precipitated total RNA (5 μl) from the peripheral blood lymphocytes on a 1% TBE agarose gel. The 28S and 18S ribosomal RNA bands are indicated and show proper quality of the RNA. B) PCR amplification of the VHH (˜700 bp) and the VH (˜900 bp) genes, as analyzed on a 1% TBE agarose gel. C) 5 μl of the digested 700 bp VHH PCR product (from B) on a 1.5% TBE agarose gel. DNA was incubated with SfiI for 7 hours at 50° C. and Eco911 for 3 hours at 37° C.

FIG. 4: Provides 10-fold dilutions of the transformed E. coli TG1 cells were spotted onto LB-agar plates supplemented with 100 μg/ml ampicillin and 2% glucose. The library size was determined by counting colonies in the highest dilution.

FIG. 5: Shows colony PCR of individual clones from each library. The insert frequency for library SNL168 day 43 is over 95%, for library SNL169 day 43 the insert frequency is 100%.

FIG. 6: Input and output from the first round of panning/selection. A). Input phages were diluted up to 1010 and used to infect E. coli TG1 before spotting on selective LB agar plates. To assess the quality of the buffers and E. coli strain used, the following TG1 were spotted: non-infect TG1 (TG1), TG1 infected with PBS used for preparing the serial dilution of inputs and outputs (PBS), TG1 infected with neutralization buffer (NB), which is Tris/HCl in this case and TG1 infected with elution buffer after neutralization (ENB), which is TEA-solution and Tris/HCl in this selection. B). Results of the selection output on wells coated with indicated amount of FimH_SI. C). Results of the selection output on wells coated with indicated amount of FimH_ST.

FIG. 7: Input and output from the second round of panning/selection. A. Input phages were diluted up to 10¹⁰ and used to infect E. coli TG1 before spotting selective LB agar plates. To assess the quality of the buffers and E. coli strain used, the following TG1 were spotted: non-infected TG1 (TG1), TG1 infected with PBS used for preparing the serial dilution of inputs and outputs (PBS), TG1 infected with neutralization buffer (NB), which is Tris/HCl in this case and TG1 infected with elution buffer after neutralization (ENB), which is TEA-solution and Tris/HCl in this selection. B. Results of the selection output on wells coated with indicated amount of FimH_SI. C. Results of the selection output on wells coated with indicated amount of FimH ST.

FIG. 8: Layout of master plate EFH-1 containing single clones for further screening in which the following controls were taken along. An empty phagemid vector (PER), an irrelevant VHH in the phagemid vector (IRR) and an empty (no bacteria) well (EM). The numbers in the plate layout correspond to the selection output from which the monoclonal VHH clones originate which is indicated in the table below the layout.

FIG. 9: Layout of master plate EFH-2 containing single clones for further screening in which the following controls were taken along. An empty phagemid vector (PER), an irrelevant VHH in the phagemid vector (IRR) and an empty (no bacteria) well (EM). The numbers in the plate layout correspond to the selection output from which the monoclonal VHH clones originate which is indicated in the table below the layout.

FIG. 10: Demonstrates binding of the periplasmic fractions of the master plate EFH-1 to FimH_SI.

FIG. 11: Demonstrates binding of the periplasmic fractions of the master plate EFH-2 to FimH_ST.

FIG. 12: Provides HinfI fingerprint of master plate EFH-1. Different digestion patterns can be distinguished.

FIG. 13: HinfI fingerprint of master plate EFH-2. The digestion patterns that can be distinguished are mostly similar but small differences are shown.

FIG. 14: Shows the sequence alignment of the clones that were picked from the selection outputs on FimH_SI (SEQ ID NOs.: 1-13 respectively, top to bottom). There is a diversity of around 10 different VHH sequences. This is based on the CDR families, and these were derived from two different germline families (KEREF and KQREL).

FIG. 15: Shows the sequence alignment of the clones that were picked from the selection outputs on FimH_ST (SEQ ID NOs.: 14-27 respectively, top to bottom). A diversity is shown of around 6 different VHH sequences. This is based on the CDR families, and these were derived from three different germline families (KEREF, KQREL and KGLEW).

FIG. 16: Coomassie stained SDS PAGE showing analysis of purified VHH selected on FimH_SI. Ref) 1 μg reference VHH, M) Marker, 1) EFH-1A1, 2) EFH-1F4, 3) EFH-1E4, 4) EFH-1C5, 5) EFH-1B2. 6) EFH-1F2, 7) EFH-1B8, 8) EFH-1D1, 9) EFH-1F8, 10) EFH-1G10. The marker that was used is the Prestained molecular weight marker (PageRuler, Thermofisher).

FIG. 17: Coomassie stained SDS PAGE showing analysis of purified VHH selected on FimH_ST. Ref) 1 μg reference VHH, M) Marker, 1) EFH-2B4, 2) EFH-2A7, 3) EFH-2D9, 4) EFH-2D7, 5) EFH-2E12, 6) EFH-2E10.

FIG. 18: Shows the binding of the VHH against FimH_SI. EFH-1D1, EFH-1F2, EFH-1E4, EFH-1F4, EFH-1C5 and EFH-1F8 show a subnanomolar apparent affinity to FimH_SI. EFH-1A1, EFH-1B2 and EFH-1G10 show a low nanomolar affinity. EFH-1B8 shows hardly any binding to FimH_SI.

FIG. 19: Shows the binding of the VHH against FimH_ST. EFH-2D7 and EFH-2E10 show a subnanomolar apparent affinity to FimH_ST.EFH-2B4 and EFH-2A7 show a low nanomolar affinity. EFH-2D9 and EFH-2E12 show a molar apparent affinity to FimH_ST.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]: “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]: “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

As used herein, the terms “colonize” and “colonization” include “temporarily colonize” and “temporary colonization”.

As used herein, “carrier”, “acceptable carrier”, or “pharmaceutical carrier” are used interchangeably and refer to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Handbook of Pharmaceutical Excipients, (Sheskey, Cook, and Cable) 2017, 8th edition, Pharmaceutical Press; Remington's Pharmaceutical Sciences, (Remington and Gennaro) 1990, 18th edition, Mack Publishing Company; Development and Formulation of Veterinary Dosage Forms (Hardee and Baggot), 1998, 2nd edition, CRC Press.

As used herein, “delivery” or “administration” means the act of providing a beneficial activity to a host. The delivery may be direct or indirect. An administration could be by an oral, nasal, or mucosal route. For example without limitation, an oral route may be an administration through drinking water, a nasal route of administration may be through a spray or vapor, and a mucosal route of administration may be through direct contact with mucosal tissue. Mucosal tissue is a membrane rich in mucous glands such as those that line the inside surface of the nose, mouth, esophagus, trachea, lungs, stomach, gut, intestines, and anus. In the case of birds, administration may be in ovo, i.e. administration to a fertilized egg. In ovo administration can be via a liquid which is sprayed onto the egg shell surface, or an injected through the shell.

As used herein, the terms “treating”, “to treat”, or “treatment”, include restraining, slowing, stopping, inhibiting, reducing, ameliorating, or reversing the progression or severity of an existing symptom, disorder, condition, or disease. A treatment may also be applied prophylactically to prevent or reduce the incidence, occurrence, risk, or severity of a clinical symptom, disorder, condition, or disease.

As used herein, “animal” includes any domesticated or non-domesticated animal, farm animal, human, or a non-human mammal. Specific examples include chickens, turkey, dogs, cats, cattle, salmon, fish, swine and horse.

In embodiments of the invention, animal includes and refers particularly to an animal susceptible to E. coli infection. In certain embodiments, animal includes and refers particularly to an animal susceptible to a bacterial gastroenteritis or urinary tract infection due to E. coli bacteria or infection.

As used herein, “gut” refers to the gastrointestinal tract including stomach, small intestine, and large intestine. The term “gut” may be used interchangeably with “gastrointestinal tract”.

As used herein, “subject” includes a human, or a non-human animal. Specific examples include chickens, turkey, dogs, cats, cattle, and swine.

The term “antibody” describes an immunoglobulin whether natural or partly or wholly synthetically produced. The term also covers any polypeptide or protein having a binding domain which is, or is homologous to, an antibody binding domain. An “antibody” is any immunoglobulin, including antibodies and fragments thereof, that binds a specific epitope. The term encompasses polyclonal, monoclonal, and chimeric antibodies. The term “antibody(ies)” includes a wild type immunoglobulin (Ig) molecule, generally comprising four full length polypeptide chains, two heavy (H) chains and two light (L) chains.

The term antibody includes and encompasses antibody fragments and domain antibodies. Antibody includes a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′) 2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv), which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody or nanobody: (vii) an isolated complementarity determining region (CDR); (viii) a Single

Chain Fv Fragment wherein a VH domain and a VL domain are linked by a peptide linker which allows the two domains to associate to form an antigen binding site (Bird et al, Science, 242, 423-426, 1988; Huston et al. PNAS USA, 85, 5879-5883, 1988): (ix) a diabody, which is a bivalent, bispecific antibody in which VH and/or VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448. (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242:193-204 9 (2000)); (xii) a minibody, which is a bivalent molecule comprised of scFv fused to constant immunoglobulin domains, CH3 or CH4, wherein the constant CH3 or CH4 domains serve as dimerization domains (Olafsen T et al (2004) Prot Eng Des Sel 17(4):315-323; Hollinger P and Hudson P J (2005) Nature Biotech 23(9):1126-1136); and (xiii) other non-full length portions of heavy and/or light chains, or mutants, variants, or derivatives thereof, alone or in any combination.

Antibody(ies) comprising linked nanobodies, such as multimeric and bi-specific versions are included in embodiments of the invention. Thus, two or more nanobodies or sequences encoding two or more nanobodies can be covalently linked, through a linker sequence or any such other recognized and applicable means, to form a bispecific or multimeric form of the nanobody(ies). In an embodiment, two distinct nanobodies are linked. In an embodiment a single nanobody is mutltimerized through linkage, which may have applicability to increase binding, avidity, affinity. In an embodiment, two or more unwue nanobodies, including nanobodies directed against distinct E. coli protein targets are linked.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains.

The term “adjuvant(s)” describes a substance, compound, agent or material useful for improving an immune response or immune cell or component stimulation, and may in some instances be combined with any particular antigen in an immunological, pharmaceutical or vaccine composition. Adjuvants can be used to increase the amount of antibody and effector T cells produced and to reduce the quantity of antigen or immune stimulant or modulator and the frequency of injection. Although some antigens are administered without an adjuvant, there are many antigens that lack sufficient immunogenicity to stimulate a useful immune response in the absence of an effective adjuvant. Adjuvants also improve the immune response from “self-sufficient” antigens, in that the immune response obtained may be increased or the amount of antigen administered may be reduced. An adjuvant can serve as a tissue depot that slowly releases the antigen and also as a lymphoid system activator that non-specifically enhances the immune response (Hood et al. Immunology, Second Ed., 1984, Benjamin/Cummings: Menlo Park, California, p. 384). In a preferred aspect an adjuvant is physiologically and/or pharmaceutically acceptable in a mammal, particularly a human. The standard adjuvant for use in laboratory animals is Freund's adjuvant. Freund's Complete adjuvant (FCA) is an emulsion containing mineral oil and killed mycobacteria in saline. Freund's incomplete adjuvant (FIA) omits the mycobacteria. Both FIA and FCA induce good humoral (antibody) immunity, and FCA additionally induces high levels of cell-mediated immunity. However, neither FCA nor FIA are acceptable for clinical use due to the side effects. In particular, mineral oil is known to cause granulomas and abscesses, and Mycobacterium tuberculosis is the agent responsible for tuberculosis. Previously known and utilized adjuvants include, but are not limited to. complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvant such as BCG (bacille Calmette-Guerin) and Corynebacterium parvum. Mineral salt adjuvants include but are not limited to: aluminum hydroxide, aluminum phosphate, calcium phosphate, zinc hydroxide and calcium hydroxide. Preferably, the adjuvant composition further comprises a lipid of fat emulsion comprising about 10% (by weight) vegetable oil and about 1-2% (by weight) phospholipids. Preferably, the adjuvant composition further optionally comprises an emulsion form having oily particles dispersed in a continuous aqueous phase, having an emulsion forming polyol in an amount of from about 0.2% (by weight) to about 49% (by weight), optionally a metabolizable oil in an emulsion-forming amount of up to 15% (by weight), and optionally a glycol ether-based surfactant in an emulsion-stabilizing amount of up to about 5% (by weight). There have been many substances that have been tried to be used as adjuvants, such as the lipid-A portion of gram negative bacterial endotoxin, and trehalose dimycolate of mycobacteria. The phospholipid lysolecithin exhibited adjuvant activity (Arnold et al., Eur. J Immunol. 9:363-366, 1979). Some synthetic surfactants exhibited adjuvant activity, including dimethyldioctadecyl ammonium bromide (DDA) and certain linear polyoxypropylenepolyoxyethylene (POP-POE) block polymers (Snippe et al., Int. Arch. Allergy Appl. Immunol. 65:390-398, 1981; and Hunter et al., J. Immunol. 127:1244-1250. 1981).

The term “specific” may be used to refer to the situation in which one member of a specific binding pair will not show any significant binding to molecules other than its specific binding partner(s). The term is also applicable where e.g. an antigen binding domain is specific for a particular epitope which is carried by a number of antigens. in which case the specific binding member carrying the antigen binding domain will be able to bind to the various antigens carrying the epitope.

The term “comprise” generally used in the sense of include, that is to say permitting the presence of one or more features or components.

The term “consisting essentially of” refers to a product, particularly a peptide sequence, of a defined number of residues which is not covalently attached to a larger product. In the case of the peptide of the invention referred to above, those of skill in the art will appreciate that minor modifications to the N- or C-terminal of the peptide may however be contemplated, such as the chemical modification of the terminal to add a protecting group or the like, e.g. the amidation of the C-terminus.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. Single letter abbreviations for amino acid residues are known in the art and one skilled in the art will recognize the amino acid each and any single letter refers to.

It should be noted that all amino-acid residue sequences are represented herein by formulae whose left and right orientation is in the conventional direction of amino-terminus to carboxy-terminus.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA. genomic DNA sequences from eukaryotic (e.g., mammalian) DNA, and even synthetic DNA sequences. A polyadenylation signal and transcription termination sequence will usually be located 3′ to the coding sequence.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art.

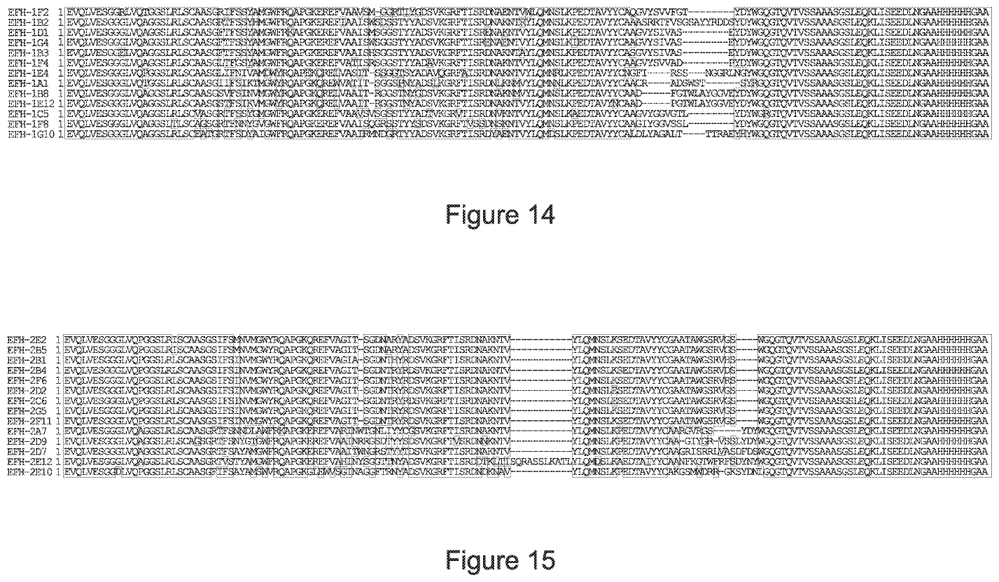

It should be appreciated that also within the scope of the present invention are DNA sequences encoding nanobodies of the invention which code for e.g. an antibody having amino acid sequence as provided herein and/or as described in FIGS. 14 and 15, or comprising the CDR domain region sequences set out herein or in FIGS. 14 and 15, but which are degenerate thereto. By “degenerate to” is meant that a different three-letter codon is used to specify a particular amino acid. It is well known in the art the codons that can be used interchangeably to code for each specific amino acid.

Mutations can be made in the sequences encoding the amino acids, nanobodies, CDR region sequences thereof including those sequences set out in FIGS. 14 and 15, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention includes sequences containing amino acid changes and substitutions, including conservative changes, which do not significantly alter the activity or binding characteristics of the resulting protein.

Exemplary and preferred conservative amino acid substitutions include any of:

glutamine (Q) for glutamic acid (E) and vice versa: leucine (L) for valine (V) and vice versa; serine(S) for threonine (T) and vice versa; isoleucine (I) for valine (V) and vice versa; lysine (K) for glutamine (Q) and vice versa: isoleucine (I) for methionine (M) and vice versa; serine(S) for asparagine (N) and vice versa; leucine (L) for methionine (M) and vice versa; lysine (L) for glutamic acid (E) and vice versa; alanine (A) for serine(S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Two amino acid sequences are “highly homologous” or “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “agent” means any molecule, including polypeptides, antibodies, polynucleotides, chemical compounds and small molecules. In particular the term agent includes compounds such as test compounds or drug candidate compounds.

The term “assay” means any process used to measure a specific property of a compound. A “screening assay” means a process used to characterize or select compounds based upon their activity from a collection of compounds.

The term “preventing” or “prevention” refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term “prophylaxis” is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Therapeutically effective amount” means that amount of a drug, compound, antimicrobial, antibody, or pharmaceutical agent that will elicit the biological or medical response of a subject that is being sought by a medical doctor or other clinician. In particular, with regard to bacterial infections and growth of bacteria, the term “effective amount” is intended to include an effective amount of a compound or agent that will bring about a biologically meaningful decrease in the amount of or extent of bacteria present in or infecting an animal. The phrase “therapeutically effective amount” is used herein to mean an amount sufficient to prevent, and preferably reduce by at least about 30 percent, more preferably by at least 50 percent. most preferably by at least 90 percent.

The term “treating” or “treatment” of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or infection, either physically, (e.g., stabilization of a discernible symptom), physiologically, (e.g., stabilization of a physical parameter), or both. In a further embodiment, “treating” or “treatment” relates to slowing the progression of a disease or reducing an infection.

The phrase “pharmaceutically acceptable” refers to molecular entities and compositions that are physiologically tolerable and do not typically produce an allergic or similar untoward reaction, such as gastric upset, dizziness and the like, when administered to a human.

Any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.” In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

Throughout this specification, quantities are defined by ranges, and by lower and upper boundaries of ranges. Each lower boundary can be combined with each upper boundary to define a range. The lower and upper boundaries should each be taken as a separate element. Two lower boundaries or two upper boundaries may be combined to define a range.

As used herein, “pg” means picogram, “ng” means nanogram, “ug” or “μg” mean microgram, “mg” means milligram, “ul” or “μl” mean microliter, “ml” means milliliter, “l” means liter.

The nanobodies of the present invention are useful in reducing or blocking E. coli colonization or infection. The invention relates to novel VHH single domain antibodies, and methods for reducing or inhibiting E. coli colonization or infection, treating bacertial gastroenteritis, Crohn's Disease, Ulcerative Colitis and urinary tract infections, and/or providing immunity against E. coli infections in animals, including humans.

The invention provides nanobodies directed against FimH antigens, selected from one or more of FimH_SI and FimH_ST. In embodiments, combinations of one or more nanobodies directed against one or more of FimH_SI and FimH_ST antigens are provided.

Selected nanobody sequences against FimH-SI are set out in Table 3 below. Sequences of the FimH-ST nanobody are as follows:

>EFH-1A1
(SEQ ID NO: 8)
EVQLVESGGGLVQTGGSLRLSCAASGIIFSTKTMGWYRQAPGKQ
REWVATLTSGGSPNYADSLKGRFTISRD
NLKNMVYLQMNNLKPEDTAVYYCAAQRADSWSTSYRGQGTQVTV
SSAAADYKDDDDKGAAHHHHHHGA
>EFH-1D1
(SEQ ID NO: 3)
EVQLVESGGGLVQAGGSLRLSCAASGFTFSSYAMGWFRKAPGKE
REFVAAISMSGGSTYYADSVKGRFTISRE
NAKNTVYLQMNSLKPEDTAVYYCAAGVYSLVASEYDYWGQGTQV
TVSSAAADYKDDDDKGAAHHHHHHG AA
>EFH-1B2
(SEQ ID NO: 2)
EVQLVESGGGLVQAGGSLRLSCAASGRTFSSYHMGWFRQAPGKE
REFIAAISWSDSSTYYADSVKGRFTISRD
NAKNTRYLQMNSLKPEDTAVYYCAAASRRTFVSGSAYYRDDSYD
YWGQGTQVTVSSAAADYKDDDDKGAA HHHHHHGAA
>EFH-1F2
(SEQ ID NO: 1)
EVQLVESGGRLVQTGGSLRLSCAASGRIFSSYAMGWFRQAPGKE
REFVAAVSMGGRTIYGDSVKGRFTISRD
NAKNTVWLQMNSLKPEDTAVYYCAQGVYSVVPGTYDYWGQGTQV
TVSSAAADYKDDDDKGAAHHHHHH GAA
>EFH-1E4
(SEQ ID NO: 7)
EVQLVESGGGLVQPGGSLRLSCAASGSIANIVAMDWYRQAPEKQ
RELVASITSSGGTSYADSVQGRFAISRD
NAKNTVYLQMNRLKPEDTAVYYCNGFIRSSNGGRLNGYWGQGTQ
VTVSSAAADYKDDDDKGAAHHHHHH GAA
>EFH-1F4
(SEQ ID NO: 6)
EVQLVESGGGLVQAGGSLRLSCAASGLTFGSYAMGWFRQAPGKE
REFVATISRSGGSTYYADAVKGRFTISRD
NAKNTVYLQMNSLKPEDTAVYYCAAGVLAVVADPYDYWGQGTQV
TVSSAAADYKDDDDKGAAHHHHH HGAA
>EFH-1C5
(SEQ ID NO: 11)
EVQLVESGGGLVQAGGSLRLSCVASGRTFSSYGMGWFRQAPGKE
REFVAAVSVSGSSTYYADTVKGRVTISR
DNVKNTVYLQMNSLKAEDTAVYYCAAGVYGGVGTLYDYWGRGTQ
VTVSSAAADYKDDDDKGAAHHHHH HGAA
>EFH-1B8
(SEQ ID NO: 9)
EVQLVESGGGLVQAGGSLRLSCAASGSVFSINVMGWYRQAPGKQ
RELVAAITRGGSTNYGDSVKGRFTISRD
NAKNMVYLQMNSLKPEDTAVYYCAADPGTWLAYGGVEYDYWGQG
TQVTVSSAAADYKDDDDKGAAHHH HHHGAA
>EFH-1F8
(SEQ ID NO: 12)
EVQLVESGGGLVQAGGSLTLSCAGSGRTFNNYGVGWFRQAPGKE
REFVAAISQGRSSTYYSDSVKGRFTVSS
DNSKNTVYLQMNSLKPEDTAVYYCAAGIYGRVSSLYDYWGQGTQ
VTVSSAAADYKDDDDKGAAHHHHHH GAA
>EFH-1G10
(SEQ ID NO: 13)
EVQLVESGGGLVQAGGSLRLSCEATGRTFSDYAIGWFRQAPGKE
REFVAAIRMNDGRTYYADSVKGRFTISR
DYAENTVYLQMDSLKPEDTAVYYCALDLYAGALTTTRAEYHYWG
QGTQVTVSSAAADYKDDDDKGAAHHH HHHGAA
Selected nanobody sequences against FimH-ST
are set out in Table 4 below. Sequences of
the FimH-ST nanobody are as follows:
>EFH-2B4
(SEQ ID NO: 17)
EVQLVESGGGLVQPGGSLRLSCAASGSIFSINVMGWYRQAPGKQ
REFVAGITSGDNTRYRDSVKGRFTISRD
NAKNTVYLQMNSLKSEDTAVYYCGAATAWGSRVDSWGQGTQVTV
SSAAADYKDDDDKGAAHHHHHHGAA
>EFH-2A7
(SEQ ID NO: 23)
EVQLVESGGGLVQAGGSLRLSCAASGRTFSNNDLAWFRKAPGKE
REFVARINWTGNLIYYQGSVKGRFTISR
DNAKNTVYLQMNSLKPEDTAVYYCAARGVRGSYDYWGQGTQVTV
SSAAADYKDDDDKGAAHHHHHHGAA
>EFH-2D7
(SEQ ID NO: 25)
EVQLVESGGGLVQAGGSLRLSCAASGRTFSAYAMGWFRQAPGKE
REFVAAITWNGRSTYYTDSVKGRFTISR
DNAKNTVYLQMNSLKPEDTAVYYCAAGRISRRLVAGDFDSWGQG
TQVTVSSAAADYKDDDDKGAAHHHH HHGAA
>EFH-2D9
(SEQ ID NO: 24)
EVQLVESGGGLVQAGGSLRLSCAGSGRTFSNYGTGWFRQAPGKE
REFVAAINRRGSDTYYSDSVKGRFTVSR
DNNKNTVYLQMNSLKPEDTAVYYCAAGIYGRVSSLYDYWGQGTQ
VTVSSAAADYKDDDDKGAAHHHHHH GAA
>EFH-2E10
(SEQ ID NO: 27)
EVQLVESGGDLVQPGGSLRLSCAASGFIFSAYAMSWVRQAPGKG
LEWVSGINAGGFTRNYADSVKGRFTISR
DNDKNAVYLQMNSLKPEDTAVYYCAKGSMWDRRGKSYDNLGQGT
QVTVSSAAADYKDDDDKGAAHHHH HHGAA
>EFH-2E12
(SEQ ID NO: 26)
EVQLVESGGGLVQAGGSLRLSCAASGRTVSTYAMGWFRQAPGKE
REFVAHINYSGGTTNYADSVKGRFTISR
DDTKLTISQRASSLKATLYLQMDSLKAEDTAIYYCAANPKGTWF
RPSDYNYWGQGTQVTVSSAAADYKDDD DKGAAHHHHHHGAA

According to the invention, the nanobodies comprise an amino acid sequence having at least 80%, at least 90%, at least 95%, at least 98%, at least 99% amino acid identity to the above sequences of nanobodies.

In one embodiment, the nanobodies comprise an amino acid sequence having at least 80%, at least 90%, at least 95%, at least 98%, at least 99% amino acid identity to the sequences of nanobodies selected from clones EFH-1DI, EFH-1F2, EFH-1E4, EFH-1F4 and EFH-1C5.

In another embodiment, combinations of one or more nanobodies directed against one or more of E. coli antigens are provided, particularly combinations of the antibodies selected and provided above.

The invention provides and relates to combinations of two or more of E. coli antigens are provided, particularly combinations of the antibodies selected and provided above. In an embodiment, the invention provides and relates to combinations of nanobodies directed to each of E. coli antigens, particularly combinations of the antibodies selected and provided above. Thus, a combination of a nanobody directed against antigen is provided. The combination may be achieved by administration of one or more or of multiple nanobodies in a single composition. The combination may be achieved by expression of one or more or of multiple nanobodies in an animal.

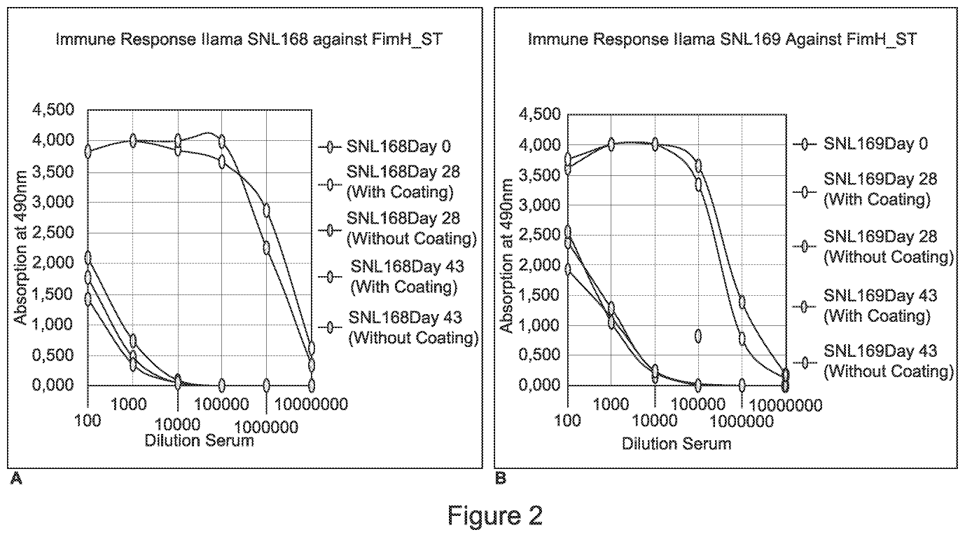

By “substantially as set out” it is meant that variable region sequences, and/or particularly the CDR sequences, of the invention will be either identical or highly homologous to the specified regions of FIG. 2, 4, 6, 8, 10 or 11. By “highly homologous” it is contemplated that only a few substitutions, preferably from 1 to 8, preferably from 1 to 5, preferably from 1 to 4, or from 1 to 3, or 1 or 2 substitutions may be made in the variable region sequence and/or in the CDR sequences. The term substantially set out as includes particularly conservative amino acid substitutions which do not materially or significantly affect the specificity and/or activity of the instant nanobodies. Conservative and non-conservative amino acid substitutions are contemplated herein for the variable region sequences and also for the CDR region sequences.

Substitutions may be made in the variable region sequence outside of the CDRs so as to retain the CDR sequences. Thus, changes in the variable region sequence or alternative non-homologous or veneered variable region sequences may be introduced or utilized, such that the CDR sequences are maintained and the remainder of the variable region sequence may be substituted.

There are several recognized and known methods and approaches to determine the CDRs in an antibody. The most commonly used CDR identification methods at present are Kabat (Wu T T, Kabat E A (1970) J Exp Med 132:211-250; Kabat EA et al (1983) Sequence of Proteins of Immunological Interest. Bethesda: National Institute of Health), IMGT (Lefranc M P et al (2003) Dev Comp Immunol 27:55-77) and Chothia (Chothia C. Lesk A M (1987) J Mol Biol 196:901-917; Chothia C et al (1989) Nature 342:877-883; Lefranc M P et al (2003) Dev Comp Immunol 27:55-77). Each of these methods has devised a unique residue numbering scheme according to which it numbers the hypervariable region residues and the beginning and ending of each of the CDRs is then determined according to certain key positions. IMGT and Kabat systems were utilized in the present studies. While these different approaches may identify slightly offset CDR sequences, they generally provide overlapping sequences and amino acids and can be useful in combination to identify amino acids which should be maintained or conserved and those that may be suitable for variation or alteration while maintaining binding. FIGS. 14 and 15 provided herein show a comparison of selected nanobodies against distinct FimH protein targets. This comparison points to regions of distinct sequence, which provides one skilled in the art direction as to the applicable heavy chain VHH CDRs and CDR1, CDR2, and CDR3 regions and sequences.

Additional residues at the N-terminal or C-terminal end of the substantial part of the variable domain may be those not normally associated with naturally occurring variable domain regions. For example, construction of specific binding members of the present invention made by recombinant DNA techniques may result in the introduction of N- or C-terminal residues encoded by linkers introduced to facilitate cloning or other manipulation steps. Other manipulation steps include the introduction of linkers to join variable domains of the invention to further protein sequences including immunoglobulin heavy chains. other variable domains (for example in the production of diabodies) or protein labels as provided herein and/or known to those of skill in the art.

The antibodies, or any fragments thereof, may be conjugated or recombinantly fused to any cellular toxin, bacterial or other, e.g. pseudomonas exotoxin, ricin, or diphtheria toxin. The part of the toxin used can be the whole toxin, or any particular domain of the toxin. Bi-and tri-specific multimers can be formed by association of different scFv molecules and have been designed as cross-linking reagents for T-cell recruitment into tumors (immunotherapy), viral retargeting (gene therapy) and as red blood cell agglutination reagents (immunodiagnostics), see e.g. Todorovska et al., J Immunol Methods. 2001 Feb. 1;248 (1-2): 47-66; Tomlinson et al., Methods Enzymol. 2000;326:461-79; McCall et al., J Immunol. 2001 May 15;166 (10): 6112-7.

Nanobodies of the invention may be labelled with a detectable or functional label. Detectable labels include, but are not limited to, radiolabels such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re, which may be attached to antibodies of the invention using conventional chemistry known in the art of antibody imaging. Labels also include fluorescent labels (for example fluorescein, rhodamine, Texas Red) and labels used conventionally in the art for MRI-CT imaging. They also include enzyme labels such as horseradish peroxidase, β-glucoronidase, β-galactosidase, urease. Labels further include chemical moieties such as biotin which may be detected via binding to a specific cognate detectable moicty, e.g. labelled avidin. Functional labels include substances which are designed to be targeted to the site of a tumor to cause destruction of tumor tissue. Such functional labels include cytotoxic drugs such as 5-fluorouracil or ricin and enzymes such as bacterial carboxypeptidase or nitroreductase, which are capable of converting prodrugs into active drugs at the site of a tumor.

As used herein. an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., MALDI mass spectrometry), array-based oligo-peptide scanning assays, and/or mutagenesis mapping (e.g., site-directed mutagenesis mapping).

In certain aspects, competition binding assays can be used to determine whether an antibody or nanobody is competitively blocked, e.g., in a dose dependent manner, by another antibody or nanobody for example, an antibody binds essentially the same epitope, or overlapping epitopes, as a reference antibody, when the two antibodies recognize identical or sterically overlapping epitopes in competition binding assays such as competition ELISA assays, which can be configured in all number of different formats, using either labeled antigen or labeled antibody. In a particular embodiment, an antibody or nanobody can be tested in competition binding assays with an antibody described herein.

Competition binding assays also can be used to determine whether two antibodies have similar binding specificity for an antigen or an epitope, including a particular epitope on an antigen or protein target. Competitive binding can be determined in an assay in which the immunoglobulin under test inhibits specific binding of another antibody to a common antigen or target antigen.

Assays known to one of skill in the art or described herein (e.g., X-ray crystallography, ELISA assays, etc.) can be used to determine if two antibodies bind to the same epitope. Biacore assays can be used to assess and determine competitive binding and also epitope binding. Biacore can be utilized to determine the extent to which different antibodies interact with a single antigen or epitope, to assess protein protein or antibody-protein interactions, and to determine binding affinity.

Immunoconjugates or antibody fusion proteins of the present invention, wherein the nanobodies of the present invention are conjugated or attached to other molecules or agents further include, but are not limited to binding members conjugated to a immunomodulator, antibacterial agent. antibiotic, or drug.

Nanobodies of the present invention may be administered to an animal in need of treatment via any suitable route, including orally, by spray administration, by injection, including intreperitoneally, intramuscularly, subcutaneous, intravenous, into the bloodstream or intestine or gut, or directly into the gut. The precise dose will depend upon a number of factors, including whether the nanobody is for diagnosis or for treatment, the dose methodology or administration type, and the applicable animal.

In an embodiment, administered comprises in ovo administration. In an embodiment, administered comprises spray administration. In an embodiment, administered comprises immersion, intranasal, intramammary. topical, or inhalation.

The compositions may further include one or more component or additive. The one or more component or additive may be a component or additive to facilitate administration. for example by way of a stabilizer or vehicle, or by way of an additive to enable administration to an animal such as by any suitable administrative means, including in aerosol or spray form, in water, in feed or in an injectable form. Administration to an animal may be by any known or standard technique. These include oral ingestion, gastric intubation, or broncho-nasal spraying. The compositions disclosed herein may be administered by immersion, intranasal. intramammary, topical, mucosally, or inhalation. When the animal is a bird the treatment may be administered in ovo or by spray inhalation.

Nanobodies of the present invention will usually be administered in the form of a pharmaceutical composition, which may comprise at least one component in addition to the specific nanbody. Thus pharmaceutical compositions or immunological compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous. or by deposition at a tumor site.

A composition of the present invention may be administered alone or in combination with other treatments, therapeutics or agents, either simultaneously or sequentially dependent upon the condition to be treated.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection. Lactated Ringer's Injection. Preservatives, stabilisers, buffers, antioxidants and/or other additives may be included, as required.

In addition, the present invention contemplates and includes therapeutic compositions for the use of the nanobody(ies) in combination with conventional antibacterial therapy. The present invention further contemplates therapeutic compositions useful in practicing the therapeutic methods of this invention. A subject therapeutic composition includes, in admixture, a pharmaceutically acceptable excipient (carrier) and one or more of a nanobody as described herein as an active ingredient.

The preparation of therapeutic compositions which contain polypeptides, analogs or active fragments as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The compositions are administered in a manner compatible with the dosage formulation, and in a therapeutically effective amount. The method and mode of administration may be adjusted or different methods and modes may be applied depending on the animal to be administered. For instance, administration in feed or spray or in water provided or applied to animals or eggs is contemplated. Administration using any of various vehicles is contemplated. Administration may include expression by virtue of an encoding plasmid, vector, nucleic acid etc. The quantity to be administered depends on the subject or animal to be treated, capacity of the subject's or animal's immune system to utilize the active ingredient, etc. Precise amounts of active ingredient required to be administered depend on the judgment of the practitioner and are peculiar to each individual. Suitable regimes for initial administration and follow on administration are also variable, and may include an initial administration followed by one or more repeated dose or doses.

Diagnostic applications of the nanobodies of the present invention, particularly antibodies and fragments thereof, include in vitro and in vivo applications well known and standard to the skilled artisan and based on the present description. Diagnostic assays and kits for in vitro assessment and evaluation of bacteria or bacterial infection or colonization may be utilized to diagnose, evaluate and monitor animal or patient samples including those known to have or suspected of being infected with E. coli or having bacterial gastroenteritis.

The present invention further provides an isolated nucleic acid encoding a nanobody of the present invention. Nucleic acid includes DNA and RNA. In a preferred aspect, the present invention provides a nucleic acid which codes for a polypeptide of the invention as defined above, including a polypeptide as provided and described herein.

The present invention also provides constructs in the form of plasmids, vectors, transcription or expression cassettes which comprise at least one polynucleotide as above. The present invention also provides a recombinant host cell which comprises one or more constructs as above. A nucleic acid encoding any specific binding member as provided itself forms an aspect of the present invention, as does a method of production of the specific binding member which method comprises expression from encoding nucleic acid therefor. Expression may conveniently be achieved by culturing under appropriate conditions recombinant host cells containing the nucleic acid. Following production by expression a specific binding member may be isolated and/or purified using any suitable technique, then used as appropriate.

Systems for cloning and expression of a polypeptide in a variety of different host cells are well known. Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate.

Thus, a further aspect of the present invention provides a host cell containing nucleic acid as disclosed herein. A still further aspect provides a method comprising introducing such nucleic acid into a host cell. The introduction may employ any available technique. The introduction may be followed by causing or allowing expression from the nucleic acid, e.g. by culturing host cells under conditions for expression of the gene. The present invention also provides a method which comprises using a construct as stated above in an expression system in order to express a specific binding member or polypeptide as above. Any of a wide variety of expression control sequences—sequences that control the expression of a DNA sequence operatively linked to it—may be used in these vectors to express the DNA sequences of this invention. A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, β-W and L-M cells, African Green Monkey kidney cells (e.g., COS 1, COS 7, BSC1, BSC40, and BMT10), insect cells (e.g., Sf9), and human cells and plant cells in tissue culture.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLES

### Example 1: Immunization/Immune Response

Purified proteins FimH_SI and FimH_ST were provided by Elanco. Immunization was carried out simultaneously for both proteins in llamas SNL168 and SNL169 at Eurogentec. Both llamas were immunized via 4 injections at day 0, 14, 28 and 35. Sera was obtained at day 0, day 28 and day 43. Peripheral blood mononuclear cells (PBMC) were obtained from a large bleed at day 43.

### Immune Response

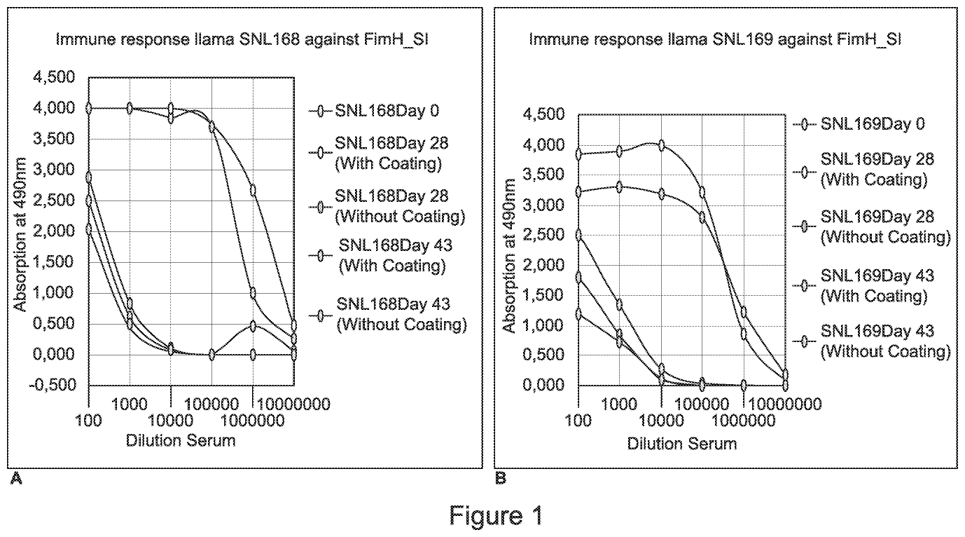

The immune response of the llamas was tested by assessing the presence of FimH_SI or FimH_ST-specific antibodies in sera of the llamas of day 0, day 28 and day 43. MaxiSorp plates were coated with 200 ng antigen per well overnight at 4° C. After three times washing with PBS containing 0.05% Tween-20 the plate was blocked with 4% milk powder in PBS (MPBS). Next, a serial dilution of the sera in 1% MPBS was added to the wells and incubated for 1 hour. Unbound antibodies were removed during washing with PBS-Tween. Subsequently, bound antibodies were detected with rabbit-anti-VHH (clone K1216) and donkey-anti-rabbit coupled to HRP. Antibody binding was quantified by the colorimetric reaction of O-phenylenediamine (OPD) in the presence of H₂O₂ at 490 nm (FIGS. 1 and 2). Both llamas show a good immune response against both FimH_SI and FimH_ST.

VHH sequences for exemplary E. coli nanobodies are provided below:

Derived protein sequences of the provided
VHH (in pMEK222)
>EFH-1A1
(SEQ ID NO: 8)
EVQLVESGGGLVQTGGSLRLSCAASGIIFSTKTMGWYRQAPGKQ
REWVATLTSGGSPNYADSLKGRFTISRD
NLKNMVYLQMNNLKPEDTAVYYCAAQRADSWSTSYRGQGTQVTV
SSAAADYKDDDDKGAAHHHHHHGAA
>EFH-1D1
(SEQ ID NO: 3)
EVQLVESGGGLVQAGGSLRLSCAASGFTFSSYAMGWFRKAPGKE
REFVAAISMSGGSTYYADSVKGRFTISRE
NAKNTVYLQMNSLKPEDTAVYYCAAGVYSLVASEYDYWGQGTQV
TVSSAAADYKDDDDKGAAHHHHHHG AA
>EFH-1B2
(SEQ ID NO: 2)
EVQLVESGGGLVQAGGSLRLSCAASGRTFSSYHMGWFRQAPGKE
REFIAAISWSDSSTYYADSVKGRFTISRD
NAKNTRYLQMNSLKPEDTAVYYCAAASRRTFVSGSAYYRDDSYD
YWGQGTQVTVSSAAADYKDDDDKGAA HHHHHHGAA
>EFH-1F2
(SEQ ID NO: 1)
EVQLVESGGRLVQTGGSLRLSCAASGRIFSSYAMGWFRQAPGKE
REFVAAVSMGGRTIYGDSVKGRFTISRD
NAKNTVWLQMNSLKPEDTAVYYCAQGVYSVVPGTYDYWGQGTQV
TVSSAAADYKDDDDKGAAHHHHHH GAA
>EFH-1E4
(SEQ ID NO: 7)
EVQLVESGGGLVQPGGSLRLSCAASGSIANIVAMDWYRQAPEKQ
RELVASITSSGGTSYADSVQGRFAISRD
NAKNTVYLQMNRLKPEDTAVYYCNGFIRSSNGGRLNGYWGQGTQ
VTVSSAAADYKDDDDKGAAHHHHHH GAA
>EFH-1F4
(SEQ ID NO: 6)
EVQLVESGGGLVQAGGSLRLSCAASGLTFGSYAMGWFRQAPGKE
REFVATISRSGGSTYYADAVKGRFTISR
DNAKNTVYLQMNSLKPEDTAVYYCAAGVLAVVADPYDYWGQGTQ
VTVSSAAADYKDDDDKGAAHHHHH HGAA
>EFH-1C5
(SEQ ID NO: 11)
EVQLVESGGGLVQAGGSLRLSCVASGRTFSSYGMGWFRQAPGKE
REFVAAVSVSGSSTYYADTVKGRVTISR
DNVKNTVYLQMNSLKAEDTAVYYCAAGVYGGVGTLYDYWGRGTQ
VTVSSAAADYKDDDDKGAAHHHHH HGAA
>EFH-1B8
(SEQ ID NO: 9)
EVQLVESGGGLVQAGGSLRLSCAASGSVFSINVMGWYRQAPGKQ
RELVAAITRGGSTNYGDSVKGRFTISRD
NAKNMVYLQMNSLKPEDTAVYYCAADPGTWLAYGGVEYDYWGQG
TQVTVSSAAADYKDDDDKGAAHHH HHHGAA
>EFH-1F8
(SEQ ID NO: 12)
EVQLVESGGGLVQAGGSLTLSCAGSGRTFNNYGVGWFRQAPGKE
REFVAAISQGRSSTYYSDSVKGRFTVSS
DNSKNTVYLQMNSLKPEDTAVYYCAAGIYGRVSSLYDYWGQGTQ
VTVSSAAADYKDDDDKGAAHHHHHH GAA
>EFH-1G10
(SEQ ID NO: 13)
EVQLVESGGGLVQAGGSLRLSCEATGRTFSDYAIGWFRQAPGKE
REFVAAIRMNDGRTYYADSVKGRFTISR
DYAENTVYLQMDSLKPEDTAVYYCALDLYAGALTTTRAEYHYWG
QGTQVTVSSAAADYKDDDDKGAAHHH HHHGAA
>EFH-2B4
(SEQ ID NO: 17)
EVQLVESGGGLVQPGGSLRLSCAASGSIFSINVMGWYRQAPGKQ
REFVAGITSGDNTRYRDSVKGRFTISRD
NAKNTVYLQMNSLKSEDTAVYYCGAATAWGSRVDSWGQGTQVTV
SSAAADYKDDDDKGAAHHHHHHGA A
>EFH-2A7
(SEQ ID NO: 23)
EVQLVESGGGLVQAGGSLRLSCAASGRTFSNNDLAWFRKAPGKE
REFVARINWTGNLIYYQGSVKGRFTISR
DNAKNTVYLQMNSLKPEDTAVYYCAARGVRGSYDYWGQGTQVTV
SSAAADYKDDDDKGAAHHHHHHGA A
>EFH-2D7
(SEQ ID NO: 25)
EVQLVESGGGLVQAGGSLRLSCAASGRTFSAYAMGWFRQAPGKE
REFVAAITWNGRSTYYTDSVKGRFTISR
DNAKNTVYLQMNSLKPEDTAVYYCAAGRISRRLVAGDFDSWGQG
TQVTVSSAAADYKDDDDKGAAHHHH HHGAA
>EFH-2D9
(SEQ ID NO: 24)
EVQLVESGGGLVQAGGSLRLSCAGSGRTFSNYGTGWFRQAPGKE
REFVAAINRRGSDTYYSDSVKGRFTVSR
DNNKNTVYLQMNSLKPEDTAVYYCAAGIYGRVSSLYDYWGQGTQ
VTVSSAAADYKDDDDKGAAHHHHHH GAA
>EFH-2E10
(SEQ ID NO: 27)
EVQLVESGGDLVQPGGSLRLSCAASGFIFSAYAMSWVRQAPGKG
LEWVSGINAGGFTRNYADSVKGRFTISR
DNDKNAVYLQMNSLKPEDTAVYYCAKGSMWDRRGKSYDNLGQGT
QVTVSSAAADYKDDDDKGAAHHHH HHGAA
>EFH-2E12
(SEQ ID NO: 26)
EVQLVESGGGLVQAGGSLRLSCAASGRTVSTYAMGWFRQAPGKE
REFVAHINYSGGTTNYADSVKGRFTISR
DDTKLTISQRASSLKATLYLQMDSLKAEDTAIYYCAANPKGTWF
RPSDYNYWGQGTQVTVSSAAADYKDDD DKGAAHHHHHHGAA

TABLE 1

| | |
|---|---|
| Target | FimH_SI/FimH_ST |
| Deliverables | Immunization 2 llamas |
| | Construction 2 phage display libraries |
| | Panning, selection and screening |
| | Sequences of at least 6 binders from at least 2 families |
| | At least 0.5 mg of purified protein of the lead clones |

Library construction

| | | |
|---|---|---|
| Llamas | SNL168 day 43 | SNL169 day 43 |
| Immune response | Good | Good |
| RNA quality | Good | Good |
| Library size | 1.3 × 10⁸ | 1.4 × 10⁸ |
| Insert frequency | >95% | 100% |

Phage display selections FimH_SI

| | | |
|---|---|---|
| Enrichment R1 | Excellent | Excellent |
| Enrichment R2 | Good | Good |

| | |
|---|---|
| Family diversity | 8 |
| Number of clones/family | 1 families of >2 clones/family |
| | 7 families of 1-2 clones/family |

| Lead | From | Relative | Apparent |
|---|---|---|---|
| candidates | family | Bmax | affinity (nM) |
| EFH-1A1 | 1 | 3.5 | 5 |
| EFH-1D1 | 2 | 3.7 | 0.1 |
| EFH-1B2 | 3 | 3.5 | 10 |
| EFH-1F2 | 4 | 4 | 0.1 |
| EFH-1E4 | 5 | 3.5 | 0.1 |
| EFH-1F4 | 2 | 3.8 | 0.1 |
| EFH-1C5 | 4 | 4 | 0.1 |
| EFH-1B8 | 6 | 2 | >400 |
| EFH-1F8 | 7 | 3.7 | 0.2 |
| EFH-1G10 | 8 | 3.6 | 7 |

Phage display selections FimH_ST

| | | |
|---|---|---|
| Enrichment R1 | Excellent | Excellent |
| Enrichment R2 | Good | Good |

| | |
|---|---|
| Family diversity | 6 |
| Number of clones/family | 1 families of >2 clones/family |
| | 5 families of 1-2 clones/family |

| Lead | From | Relative | Apparent |
|---|---|---|---|
| candidates | family | Bmax | affinity (nM) |
| EFH-2B4 | 1 | 3.5 | 6 |
| EFH-2A7 | 2 | 3.2 | 6 |
| EFH-2D7 | 3 | 3 | 0.4 |
| EFH-2D9 | 4 | 1.1 | >40 |
| EFH-2E10 | 5 | 3.5 | 0.5 |
| EFH-2E12 | 6 | 3.2 | >70 |

Comments
Both animals showed a good immune response. The library construction was satisfactory resulting in libraries with a good number of transformants. Both rounds of selection resulted in good phage output titers and a broad range of clones. Screening and sequencing has elucidated 10 clones with high potential. EFH-1D1, EFH-1F2, EFH-1E4, EFH-1F4, EFH-1C5 and EFH-1F8 bind very good to FimH_SI. EFH-1A1, EFH-1B2 and EFH-1G10 bind well to FimH_SI. Clone EFH-1B8 should be considered as not such a good binder to FimH_SI. Clones EFH-1D1, EFH-1F2, EFH-1E4, EFH-1F4 and EFH-1C5 should be considered as lead candidate, based on affinity binding and production level in E. coli.

### Example 2: Library construction of SNL168 day 43 and SNL169 day 43

RNA Isolation and cDNA Synthesis

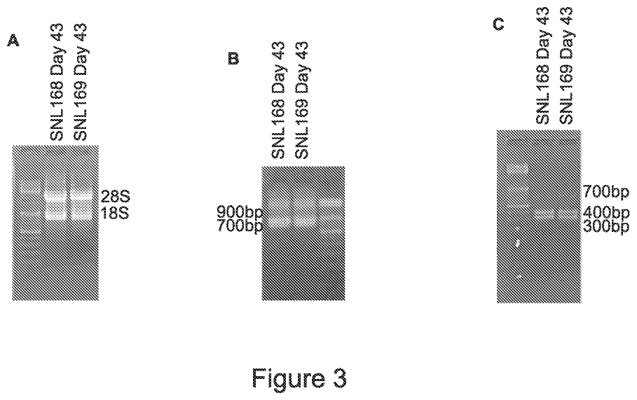

Peripheral blood lymphocytes were isolated from a large bleed at day 43 from which RNA was isolated at Eurogentec. Precipitated RNA was dissolved in RNase-free MQ and the RNA concentrations were measured. To assess the quality of the RNA, Sul of the dissolved RNA was analyzed on gel. FIG. 3A shows that intact 28S and 18S rRNA was clearly visible, indicating proper integrity of the RNA.

About 40 μg RNA (4 reactions of 10 μg each) was transcribed into cDNA using a reverse transcriptase Kit (Invitrogen). The cDNA was purified on Macherey Nagel PCR clean-up columns. Variable domains of the heavy chains (both conventional and heavy chain-only) fragments were amplified using primers annealing at the leader sequence region and at the CH2 region. 5 μl was loaded onto a 1% TBE agarose gel for a control of the amplification. FIG. 3B shows that the two DNA fragments ˜700 bp (for VHH+hinge) and ˜900 bp (VH+CH1+hinge) were amplified representing the VHH and VH, respectively.

After this control, the remaining of the samples were loaded on a 1% TAE agarose gel and the 700 bp fragment was excised and purified from the gel. A total of 80 ng of isolated PCR product was used as a template for the nested PCR (end volume 800 μl) to introduce SfiI and Eco91I restriction sites to either end of the VHH gene. The amplified VHH fragment was cleaned on Macherey Nagel PCR cleaning columns and eluted in 120 μl. The eluted DNA was first digested with SfiI, followed by Eco91I. As a control of the restriction digestion, 4 μl of this mixture was loaded onto a 1.5% TBE agarose gel. FIG. 3C shows that the DNA has been properly digested and the 400 bp VHH band and the remaining 300 bp (hinge) bands are clearly visible.

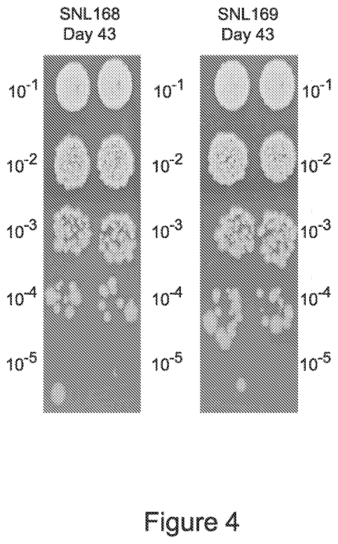

After the restriction digestion, the samples were loaded on a 1.5% TAE agarose gel. The 400 bp fragment was excised from the gel and purified on Machery Nagel gel extraction columns. The purified 400 bp VHH fragments (˜330 ng) were ligated into the pUR8100 phagemid vector (˜1 μg) and transformed into TG1 E. coli. The transformed TG1 were titrated using 10-fold dilutions. 5 μl of the dilutions were spotted on LB-agar plates supplemented with 100 μg/ml ampicillin and 2% glucose (FIG. 4)

The number of transformants was calculated from the spotted dilutions of the transformed TG1 culture (keeping in mind that the final volume of the transformation is 8 ml) (FIG. 4). The total number of transformants and thereby the size of the library was calculated by counting colonies in the highest dilution and using the formula below:

Library size=(amount of colonies)*(dilution)*8 (ml)/0.005 (ml; spotted volume)

Table 2 shows the calculated library size, including the measured OD₆₀₀ of the culture. All libraries were of good size with more than 10⁸ clones per library. The bacteria were stored in 2xYT medium supplemented with 20% glycerol, 2% glucose and 100 μg/ml ampicillin at −80° C.

TABLE 2
The size (# of transformants) of the libraries
and the measured OD₆₀₀ of the glycerol stocks.

| Llama | Library size | OD₆₀₀ |
|---|---|---|
| SNL168 day 43 | 1.28 × 10⁸ | 89.6 |
| SNL169 day 43 | 1.44 × 10⁸ | 84.0 |

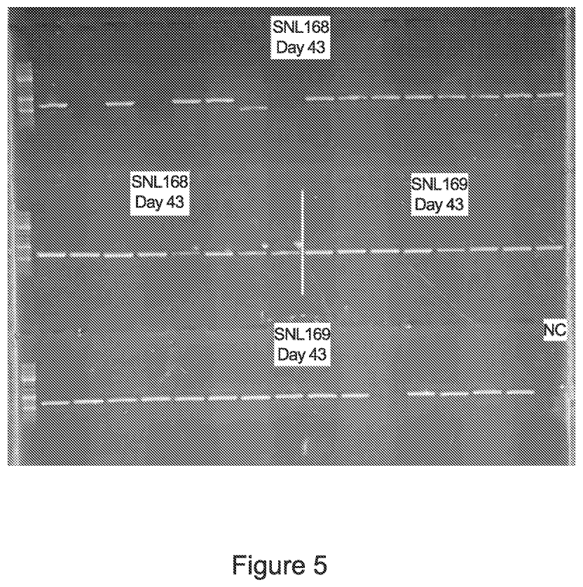

The VHH insert frequency in the phagemid vector was determined by picking 24 different clones from each transformed library and performing a colony PCR. Bands of ˜700 bp indicate a successfully cloned VHH fragment. Bands of ˜300 bp indicate an empty plasmid. The insert frequency for library SNL168 day 43 is over 95%, for library SNL169 day 43 the insert frequency is 100% (FIG. 5).

### Example 3: Phage Production and Selection

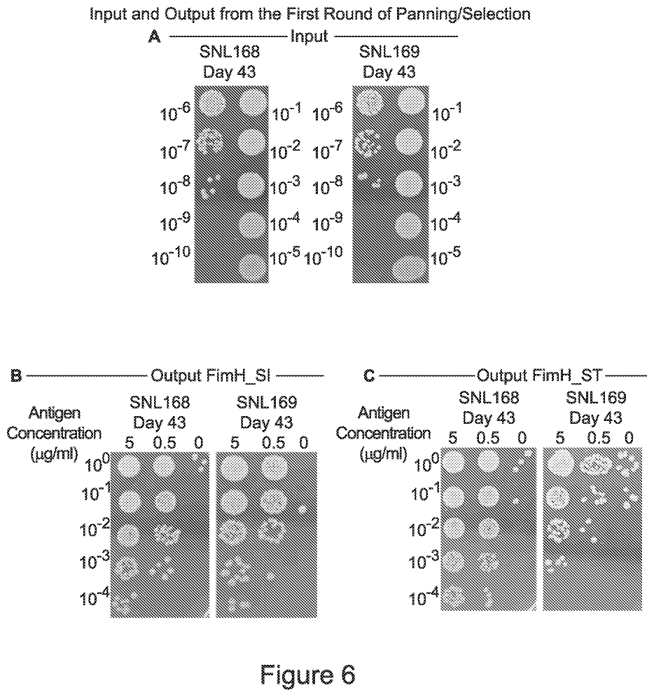

Phages were produced from the libraries as outlined below: E. coli TG1 containing libraries SNL168 day 43 and SNL169 day 43 were diluted from the glycerol stock up to an OD600 of 0.05 in 2xYT medium containing 2% glucose and 100 μg/ml ampicillin. The number of bacteria in this inoculum was at least 10× the library size (>108 bacteria inoculum). This culture was grown at 37° C. for 2 hours to reach an OD600 of ˜0.5. Subsequently, about 7 ml of the cultures were infected with helper phage VCS M13 using a MOI (multiplicity of infection) of 100 for 30 minutes standing at 37° C. Infected bacteria were spun down and resuspended into 50 ml fresh 2xYT medium supplemented with both ampicillin (100 μg/ml, for the phagemid) and kanamycin (25 μg/ml, for the M13 phage) and grown overnight at 37° C., shaking. Produced phages were precipitated from the supernatant of the cultures using PEG-NaCl precipitation. Titers of the produced phages were calculated by serial dilution of the phage and infection of E. coli TG1. Titers of the libraries were 5x1011/ml for SNL168 day 43 and 5x1011/ml for SNL169 day 43, respectively (see FIG. 6A), which was sufficient for continuing with the selections.

For the 1st round of panning/selections, 20 μl of the precipitated phages (˜1011 phages, which is >200-fold the diversity of the libraries) were applied to wells coated with FimH_SI or FimH_ST. In short, for each library, 100 μl antigen was coated on the MaxiSorp plate overnight at 2 concentrations 5 μg/ml and 0.5 μg/mL. As a negative control, one well was incubated with PBS only. Next day after removal of non-bound antigen, the plate was washed three times with PBS and blocked with 4% milk powder in PBS (MPBS). At the same time freshly precipitated phages were pre-blocked with 2% MPBS for 30 minutes. Pre-blocked phages were incubated with directly coated FimH_SI or FimH_ST for 2 hours. Upon extensive washing with PBS-Tween and PBS, bound phages were eluted with 0.1M TEA-solution, which was subsequently neutralized with 1M Tris/HCl pH 7.5. Eluted phages were serially diluted and then used to infect TG1 bacteria and spotting on LB-agar plates supplemented with 2% glucose and 100 μg/ml ampicillin and incubated overnight at 37° C. (FIG. 6B).

FIG. 6 shows that for both libraries very high levels of binding phages were eluted from the coated wells, for both FimH_SI (FIG. 6B) and FimH_ST (FIG. 6C). Also a concentration dependent enrichment between the different concentrations of antigen used in the panning was observed. Very little non-specific binding phages were eluted from the non-coated wells. All eluted phages (output) were infected E. coli TG1 (rescue), which were stored at −80° C. as glycerol stock. For the 2nd round of selection, new phages were produced of rescued outputs from the selection on 5 μg/ml FimH_SI or FimH_ST (highest coating) Overnight grown rescued outputs were diluted 100-fold in 5ml fresh 2xYT medium supplemented with 2% glucose and 100 μg/ml ampicillin and grown for 2 hours until log-phase. Subsequently 1 μl of helper phage VCS M13 was added and incubated at 37° C. for 30 minutes. Cultures were allowed to produce phages overnight at 37° C. Produced phages were precipitated from the supernatant of the cultures using PEG-NaCl precipitation.

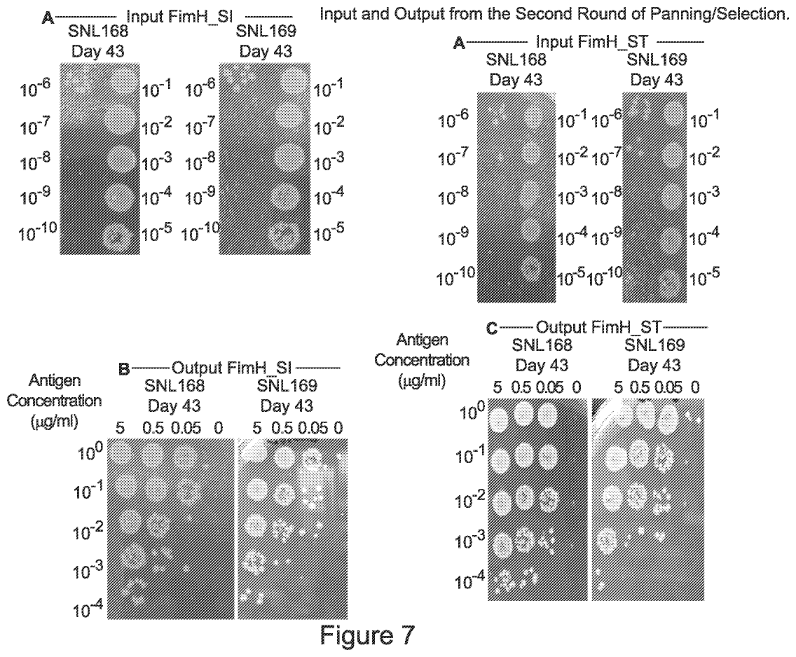

For the 2nd round of panning/selection, 1 μl of the precipitated phages was applied to wells coated with FimH_SI or FimH_ST as indicated below: antigen was coated on the MaxiSorp plate overnight at 3 concentrations (5 μg/ml, 0.5 μg/ml and 0.05 μg/ml). As a negative control, one well was incubated with PBS only. Next day, after removal of non-bound antigen, the plate was washed three times with PBS and blocked with 4% MPBS. At the same time freshly precipitated phages were pre-blocked in 2% MPBS for 30 minutes as described above. Pre-blocked phages were incubated with directly coated FimH_SI or FImH_ST for 2 hours. Phage libraries were incubated with the same coated antigen as they were derived from in the first round of panning. Upon extensive washing with PBS-Tween and PBS, bound phages were eluted with 0.1M TEA-solution and subsequently neutralized with 1M Tris/HCl pH7.5. Eluted phages were serially diluted and then used to infect TG1 cells and spotting on LB-agar plates supplemented with 2% glucose and 100 μg/ml ampicillin and incubated overnight at 37° C. (see FIG. 7).

As shown in FIG. 7, again, very high outputs were eluted from the coated wells, for both antigens, showing a concentration dependent enrichment between the different concentrations used. Hardly any aspecific binding phages were eluted from the non-coated wells. This suggests that VHH binding specifically to FimH_SI or FimH_ST were selected. After the 2^{nd} round of phage display selection, phages were rescued by infection of E. coli TG1, glycerol stocks were prepared.

### Example 4: Screening After 2 Rounds of Phage Display Selections

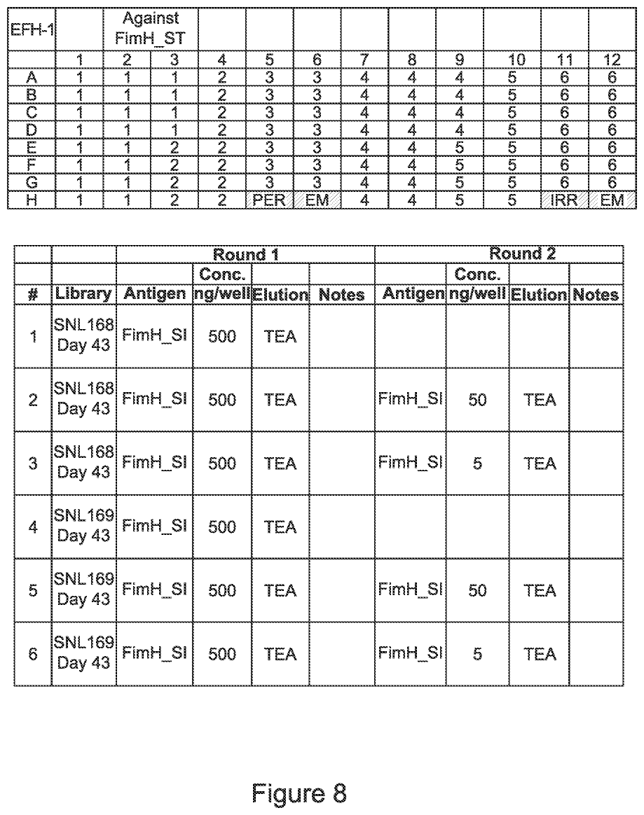

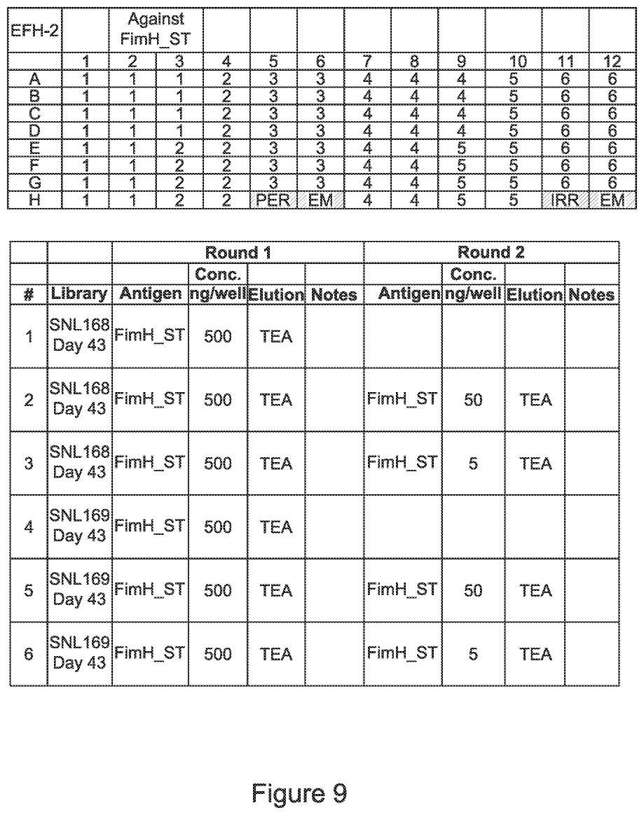

Rescued outputs of the 1^{st} and of the 2^{nd} round of selection on FimH_SI and FimH_ST were plated out in order pick single clones. For both master plate EFH-1 and EFH-2, a total of 92 single clones were picked in a 96-wells plate according to the schemes indicated in FIG. 8 and FIG. 9. In order to screen master plate EFH-1 for FimH_SI-binders and master plate EFH-2 for FimH_ST-binders, periplasmic extracts containing monoclonal VHH were produced. The master plate was cultivated at 37° C. in 2xYT medium supplemented with 2% glucose and 100 μg/ml ampicillin and stored at −80° C. after addition of glycerol to a final concentration of 20%. For the production of periplasmic extracts, master plates EFH-1 and EFH-2 were duplicated into a deep well plate containing 1ml 2xYT medium supplemented with 0.1% glucose and 100 μg/ml ampicillin and grown for 3 hours at 37° C. before adding 1 mM IPTG for induction of VHH expression. The VHH expression was conducted overnight at room temperature.

Periplasmic extracts were prepared by collecting the bacteria by centrifugation, resuspension of this pellet into 120 μl PBS and one freeze-thaw cycle. Bacteria were centrifuged to separate the soluble periplasmic fraction containing the VHH from the cell debris (pellet).

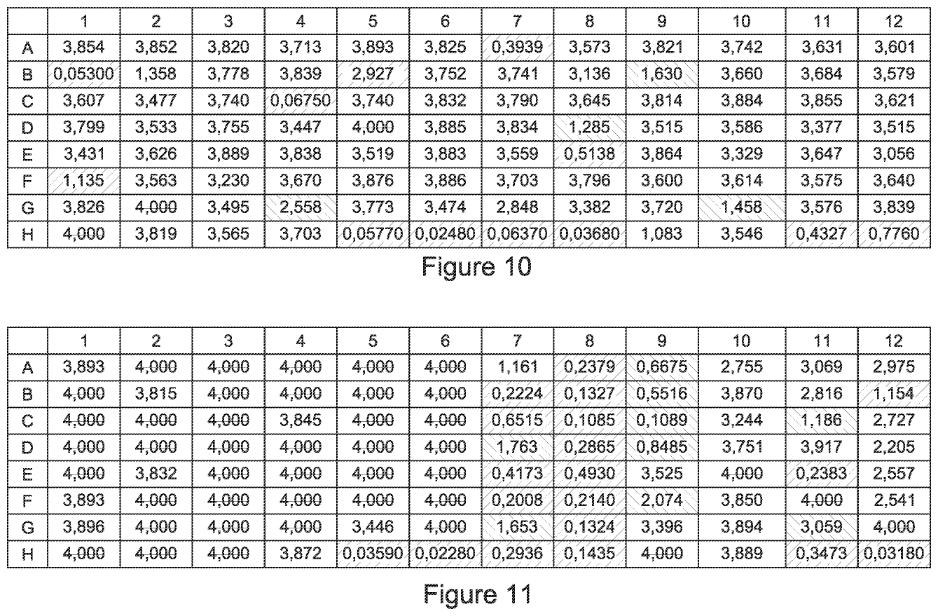

To test the binding specificity of the monoclonal VHH by ELISA, FimH_SI and FimH_ST (100 ng/well in PBS) were coated overnight onto MaxiSorp plates at 4° C. The coated plates were washed and subsequently blocked using 4% MPBS. The blocked wells were incubated with 10 μl of the periplasmic extracts and 40 μl 1% MPBS for 1 hour at room temperature. Unbound VHH were removed by washing with PBS containing 0.05% Tween-20. Subsequently, bound VHH were detected with rabbit-anti-VHH (clone K976) and donkey-anti-rabbit coupled to HRP. Binding of the VHH was quantified by the colorimetric reaction of OPD in the presence of H₂O₂ at 490 nm (FIGS. 10 and 11). FIG. 10 shows that most of the clones of master plate EFH-1 were able to bind specifically to FimH_SI. FIG. 11 shows that also most clones of master plate EFH-2 were able to bind specifically to FimH_ST. For the first round selection output from library SNL169 day 43 on 5 μg/ml FimH_ST, less binding clones are detected in the master plate (columns 7, 8 and partially 9). The outputs from the same library in the second round of panning (columns 10 till 12) do show a lot of clones binding to FimH_ST, indicating that in the second round of panning, better binding VHH were selected.

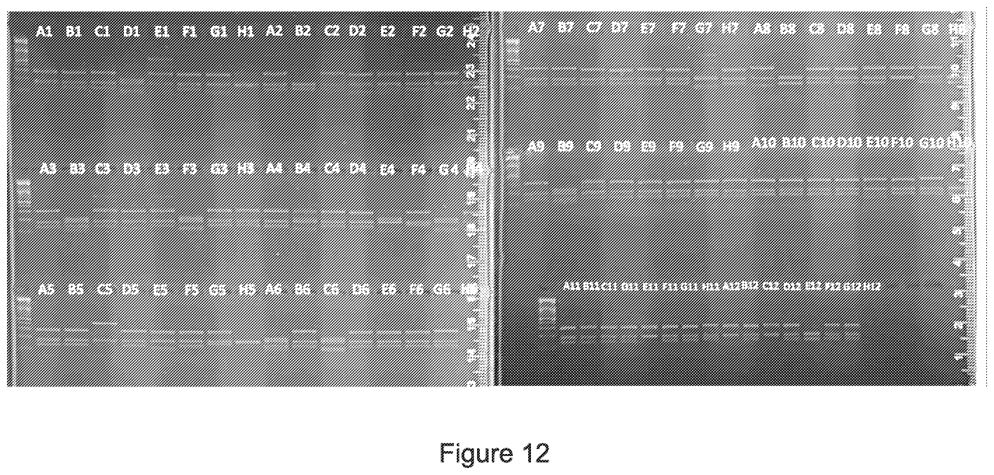

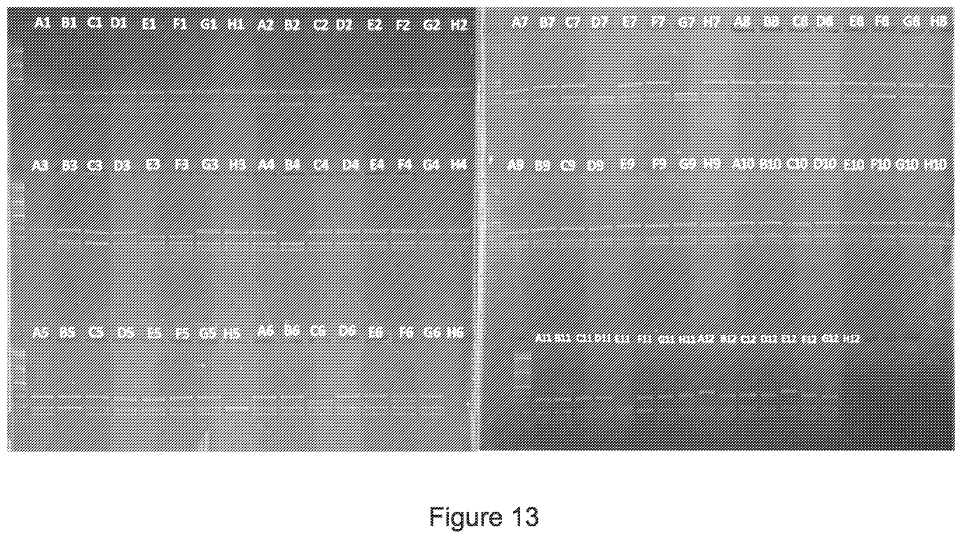

To assess the diversity of clones selected, the VHH insert of the different clones were amplified with PCR and subsequently digested with HinfI to generate a HinfI fingerprint pattern (FIGS. 12 and 13). FIGS. 12 and 13 show that different HinfI fingerprint pattern are obtained from the different VHH after the HinfI digestion, indicating that clones with different VHH sequences are present in the master plate

### Example 5: Sequence Analysis of FimH_SI and FimH_ST Binding VHH

Based on the ELISA results (FIGS. 10 and 11) and the HinfI digestion patterns (FIGS. 12 and 13), 13 clones for FimH_SI (EFH-1A1, EFH-1D1, EFH-1B2, EFH-1F2, EFH-1B3, EFH-1E4, EFH-1F4, EFH-1G4, EFH-1C5, EFH-1B8, EFH-1F8, EFH-1G10 and EFH-1E12) and 14 clones for FimH_ST (EFH-2B1, EFH-2D2, EFH-2E2, EFH-2B4, EFH-2B5, EFH-2G5, EFH-2C6, EFH-2F6, EFH-2A7, EFH-2D7, EFH-2D9, EFH-2E10, EFH-2F11 and EFH-2E12) were selected for sequence determination. In principle, such a panel should provide a good representation of the clones present in the elution of the different selection outputs.

FIG. 14 shows the sequence alignment of the clones that were picked from the selection outputs on FimH_SI. There is a diversity of around 10 different VHH sequences. This is based on the CDR families, and these were derived from two different germline families (KEREF and KQREL).

FIG. 15 shows the sequence alignment of the clones that were picked from the selection outputs on FimH_ST. A diversity is shown of around 6 different VHH sequences. This is based on the CDR families, and these were derived from three different germline families (KEREF, KQREL and KGLEW).

### FIG. 6: Cloning and Production of VHH Selected on FimH_SI and FimH_ST

From all the sequenced clones, 10 clones against FimH_SI (EFH-1A1, EFH-1D1, EFH-1B2, EFH-1F2, EFH-1E4, EFH-1F4, EFH-1C5, EFH-1B8, EFH-1F8 and EFH-1G10) and 6 clones against FimH_ST (EFH-2B4, EFH-2A7, EFH-2D7, EFH-2D9, EFH-2E10 and EFH-2E12) were subcloned from the phagemid vector into the expression vector pMEK222 using SfiI and Eco91I restriction enzymes. Recloning into pMEK222 also adds a FLAG and His-tag to the C-terminus of the VHH, allowing detection and affinity purification.

For the production, pre-cultures were prepared by growing the bacteria containing the plasmids with the selected VHH in 8 ml 2xYT medium supplemented with 2% glucose and 100 μg/ml ampicillin overnight at 37° C. The pre-cultures were diluted into 800 ml fresh 2xYT that was pre-warmed at 37° C. and supplemented with 100 μg/ml ampicillin and 0.1% glucose. The bacteria were grown for 2 hours at 37° C. before induction of the VHH expression with 1 mM IPTG. The VHH were expressed for 4 hours at 37° C. and bacteria were harvested by centrifugation. Bacteria pellets were resuspended into 30 ml PBS and frozen at −20°.

### FIG. 7: Purification and Analysis of the VHH

Frozen bacteria pellets were thawed at room temperature and cell debris was spun down by centrifugation. VHH were purified from the supernatant (soluble fraction) using affinity of the His-tag to Cobalt charged sepharose beads (Immobilized Metal Affinity Chromatography (IMAC) using TALON beads). Bound VHH were eluted with 150 mM imidazole and dialyzed against PBS.

The protein concentration was measured using absorption at 280 nm and corrected according the molar extinction coefficient and the molecular weight of the different VHH (Tables 3 and 4).

TABLE 3
Concentrations of the produced VHH selected on
FimH_SI. Concentrations were calculated using
the absorption (A280) and the correction factor (CF).

| | | | | | | Production |
|---|---|---|---|---|---|---|
| | | | | | | level |
| VHH | Vector | Tag | CF | MW | A280 | (mg/L) |
| EFH-1A1 | pMEK222 | FLAG-His | 1.775 | 15240 | 1.546 | 1.306 |
| EFH-1D1 | pMEK222 | FLAG-His | 1.689 | 15405 | 4.850 | 4.306 |
| EFH-1B2 | pMEK222 | FLAG-His | 1.978 | 16694 | 4.210 | 3.193 |
| EFH-1F2 | pMEK222 | FLAG-His | 1.844 | 15479 | 5.926 | 4.820 |
| EFH-1E4 | pMEK222 | FLAG-His | 1.403 | 15359 | 2.330 | 3.320 |
| EFH-1F4 | pMEK222 | FLAG-His | 1.606 | 15282 | 2.840 | 2.653 |
| EFH-1C5 | pMEK222 | FLAG-His | 1.702 | 15292 | 6.408 | 5.648 |
| EFH-1B8 | pMEK222 | FLAG-His | 1.929 | 15574 | 5.980 | 4.651 |
| EFH-1F8 | pMEK222 | FLAG-His | 1.689 | 15411 | 0.618 | 0.549 |
| EFH-1G10 | pMEK222 | FLAG-His | 1.690 | 16278 | 4.670 | 4.144 |

TABLE 4
Concentrations of the produced VHH selected on
FimH_ST. Concentrations were calculated using
the absorption (A280) and the correction factor (CF).

| | | | | | | Production |
|---|---|---|---|---|---|---|
| | | | | | | level |
| VHH | Vector | Tag | CF | MW | A280 | (mg/L) |
| EFH-2B4 | pMEK222 | FLAG-His | 1.695 | 15080 | 4.850 | 4.291 |
| EFH-2A7 | pMEK222 | FLAG-His | 1.854 | 15400 | 7.192 | 5.820 |
| EFH-2D7 | pMEK222 | FLAG-His | 1.713 | 15792 | 1.420 | 1.243 |
| EFH-2D9 | pMEK222 | FLAG-His | 1.666 | 15620 | 3.120 | 2.809 |
| EFH-2E10 | pMEK222 | FLAG-His | 1.731 | 15630 | 3.700 | 3.206 |
| EFH-2E12 | pMEK222 | FLAG-His | 1.766 | 17012 | 3.700 | 3.143 |

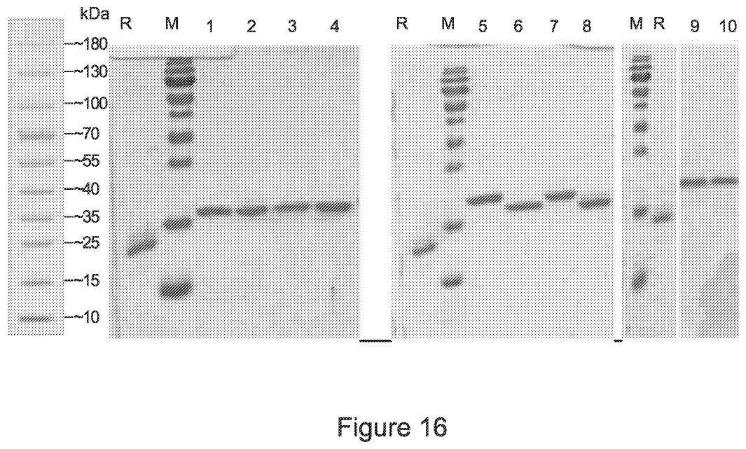

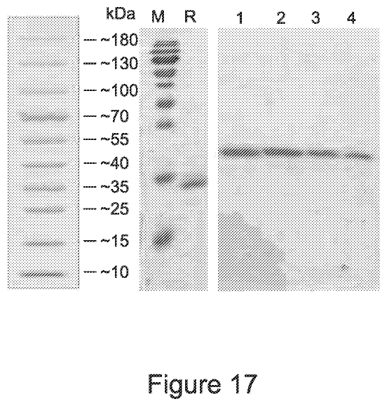

As a quality check, 1 μg of purified VHH was loaded on a SDS-PAGE (FIGS. 16 and 17). FIGS. 16 and 17 show that all VHH show appropriate purity.

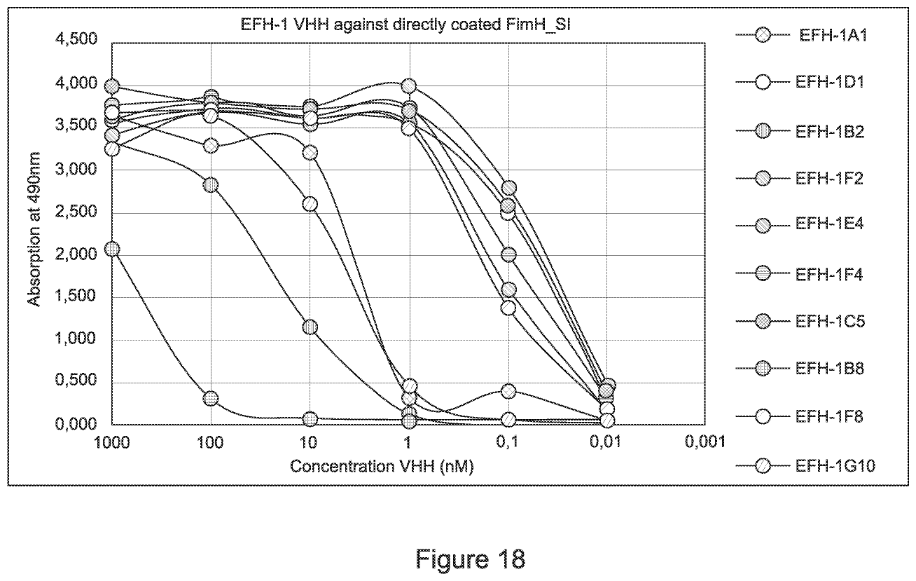

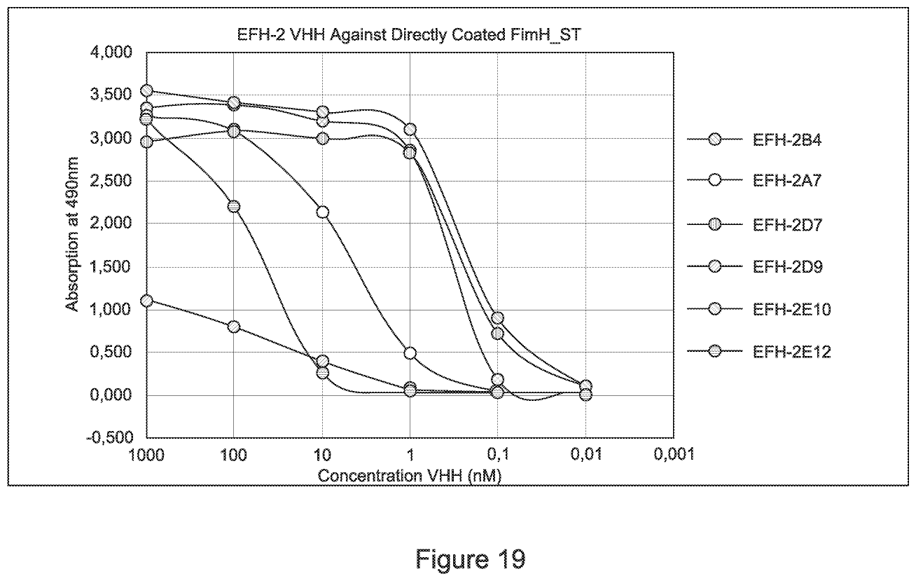

The binding of purified VHH to immobilized FimH_SI or FimH_ST was analyzed by ELISA. MaxiSorp plates were coated with 200 ng/well antigen overnight at 4° C. in PBS. After blocking the wells with 4% MPBS, a serial dilution of the VHH was added to the coated wells and incubated for 1 hour at room temperature. After washing unbound VHH, bound VHH were detected using a rabbit-anti-VHH (clone K976) and donkey-anti-rabbit coupled to HRP. Binding was quantified by measuring colorimetric reaction of OPD+H₂O₂ at 490 nm (FIGS. 18 and 19).

The binding of the VHH against FimH_SI is shown in FIG. 18. EFH-1DI, EFH-1F2, EFH-1E4, EFH-1F4, EFH-1C5 and EFH-1F8 show a subnanomolar apparent affinity to FimH_SI. EFH-1A1, EFH-1B2 and EFH-1G10 show a low nanomolar affinity. EFH-1B8 shows hardly any binding to FimH_SI.

FIG. 19 shows the binding of the VHH against FimH_ST. EFH-2D7 and EFH-2E10 show a subnanomolar apparent affinity to FimH_ST.EFH-2B4 and EFH-2A7 show a low nanomolar affinity. EFH-2D9 and EFH-2E12 show a molar apparent affinity to FimH_ST.

In conclusion, immunization of the two llamas with FimH_SI and FimH_ST resulted in a good immune response. The generated libraries were of a good size and insert frequency. Phage display selections on FimH_ST has resulted in a number of good clones of which EFH-2D7 and EFH-2E10 show a very good apparent affinity and high production level in E. coli and should therefore be considered as the lead candidates. For FimH_SI can be concluded that EFH-1D1, EFH-1F2, EFH-1E4, EFH-1F4 and EFH-1C5 show an excellent apparent affinity and very high production levels in E. coli. Therefore, these should be considered as lead candidates for FimH_SI.

This invention may be embodied in other forms or carried out in other ways without departing from the spirit or essential characteristics thereof. The present disclosure is therefore to be considered as in all aspects illustrated and not restrictive, the scope of the invention being indicated by the description hereof, and all changes which come within the meaning and range of equivalency are intended to be embraced therein.

Any references a cited throughout this Specification are herein incorporated by reference in their entirety.

1. A polypeptide selective for type 1 fimbriae D-mannose specific adhesin (FimH), wherein said polypeptide comprises a sequence selected from EFH-2D7 (SEQ ID NO:25) and EFH-2E10 (SEQ ID NO:27).

2. A method for reducing or inhibiting E. coli colonization or infection in a host, comprising administering to said host a composition comprising a polypeptide selective for type 1 fimbriae D-mannose specific adhesin (FimH).

3. The method according to claim 2, wherein the polypeptide comprises a sequence selected from EFH-2D7 (SEQ ID NO:25) and EFH-2E10 (SEQ ID NO:27)

4. The method according to claim 2, wherein the host suffers from bacterial gastroenteritis, Crohn's Disease, ulcerative colitis and/or urinary tract infections.

5. A method for enhancing an immune response against E. coli infections in animals, comprising administering to said host a composition comprising a polypeptide selective for type 1 fimbriae D-mannose specific adhesin (FimH).

6. The method according to claim 5, wherein the polypeptide comprises a sequence selected from EFH-2D7 (SEQ ID NO::25) and EFH-2E10 (SEQ ID NO::27)
